SEQUENCE LISTING (13 sequences)

SEQ ID NO: 1 - RNA, 19 nt, Artificial
  siRNA
ggacggcaauacuacuuua

SEQ ID NO: 2 - RNA, 19 nt, Artificial
  siRNA
agcucgagagagcauuaca

SEQ ID NO: 3 - RNA, 19 nt, Artificial
  siRNA
acagcuugagagagcuuua

SEQ ID NO: 4 - protein, 23 aa, Artificial
  motif
GLHLLHXQHFXHGHXNIPPVTLA

SEQ ID NO: 5 - protein, 8 aa, Artificial
  motif
FLXPXKPL

SEQ ID NO: 6 - protein, 21 aa, Artificial
  motif
DWXRHLLSPHHHXDDHHLYFN

SEQ ID NO: 7 - protein, 8 aa, Artificial
  motif
LWKGHXLE

SEQ ID NO: 8 - protein, 10 aa, Artificial
  motif
FSLXLXGHVY

SEQ ID NO: 9 - protein, 23 aa, Artificial
  motif
CAVGFSGVLFXLKVXXNXYXPGG

SEQ ID NO: 10 - protein, 10 aa, Artificial
  motif
ACWHELHHIH

SEQ ID NO: 11 - protein, 24 aa, Artificial
  motif
PGTSFHGHXXGILVGLHYTXGPLK

SEQ ID NO: 12 - protein, 10 aa, Artificial
  motif
YTXGHXEEEQ

SEQ ID NO: 13 - protein, 12 aa, Artificial
  motif
EEHRRXRHXRFD

(19) United States
(12) Patent Application Publication
(10) Pub. No.: US 2010/0173972 A1
(43) Pub. Date: Jul. 8, 2010

(54) METHODS AND USES RELATED TO RHBDL4

(75) Inventors: Marius Kasper Lemberg, Cambrigde (GB); Matthew Freeman, Cambrigde (GB)

Correspondence Address: NIXON PEABODY, LLP, 401 9TH STREET, NW, SUITE 900, WASHINGTON, DC, 20004-2128, US

(73) Assignee: MEDICAL RESEARCH COUNCIL, London (GB)

(21) Appl. No.: 12/595,367

(22) PCT Filed: Apr. 11, 2008
(86) PCT No.: PCT/GB2008/001295

§ 371 Date: Dec. 16, 2009

Related U.S. Application Data
(60) Provisional application No. 60/911,303, filed on Apr. 12, 2007.

(30) Foreign Application Priority Data
Apr. 12, 2007 (GB) 0707069.1

(51) Int. Cl. A61K 31/713; C07H 21/02; A61P 13/12; A61P 9/00; A61P 35/00; C12Q 1/34

(52) U.S. Cl. 514/44 A; 536/24.1; 435/18; 435/212

(57) ABSTRACT

The invention relates to a method of identifying a modulator of RHBDL4, said method comprising (i) providing a first and second sample of cells; (ii) contacting said first sample of cells with a candidate modulator of RHBDL4; (iii) measuring epidermal growth factor receptor (EGFR) transactivation in said first and second samples of cells, wherein a difference between the transactivation measured in said first and second samples of cells identifies said candidate modulator of RHBDL4 as a modulator of RHBDL4. The invention also relates to RHBDL4 protease assays and to uses of RHBDL4 protease and methods of cleavage of RHBDL4 substrates.

### FIELD OF THE INVENTION

The invention relates to certain rhomboid family serine proteases and to their uses and to assays for assessing their action and/or activities. In particular the invention relates to RHBDL4 type rhomboids.

### BACKGROUND TO THE INVENTION

EGFR signaling in mammals regulates multiple developmental decisions and in humans its hyperactivity underlies may pathologies, including cancer. Genetic studies in model organisms have revealed the importance of rhomboid intramembrane proteases in EGFR control. For example, rhomboids are the cardinal regulators of EGFR signalling in Drosophila. Given the general conservation of signaling pathways, it has been a mystery that mammalian EGFR signalling has been found to be rhomboid independent.

Drosophila rhomboids can function by releasing membrane tethered EGF-like growth factors, allowing them to activate the EGFR in neighboring cells. Despite this key activity, there has been no evidence for mammalian rhomboids having a similar role.

Since EGF receptor signalling plays a part in many human diseases as well as in development, it is clearly important to understand its physiological regulation. TGFα, the most biologically significant EGFR ligand, is activated by proteolytic cleavage, releasing it from the signal emitting cell. This release requires ADAM metalloproteases like TACE.

WO 02/093177 discloses various members of the rhomboid family, in particular the Drosophila rhomboid family. It is noted on page 8 of this document that a polypeptide which is a member of the rhomboid family shares greater than 18% sequence identity with the sequence of Drosophila Rhomboid-1 at the amino acid level, and/or shares greater than 30% sequence similarity to Drosophila Rhomboid-1 at the amino acid level. There is no disclosure of nor mention of RHBDL4 in this document.

Koonin et al (Genome Biology 2003 Volume 4 Article R19) discloses that the rhomboids are a nearly ubiquitous family of intramembrane serine proteases. The results disclosed in this document are based purely on insilico analysis. There is no experimental demonstration of any function for any rhomboid in this document. This document mentions the mouse equivalent of RHBDL4. This is mentioned as one of hundreds of individual possible rhomboids upon which the sequence analysis was conducted. This mouse rhomboid was classified as a mitochondrial rhomboid.

The present invention seeks to overcome problem(s) associated with the prior art.

### SUMMARY OF THE INVENTION

The present inventors have undertaken a comprehensive evolutionary study of the rhomboid family. This has been based not only on sequence analysis, but also on phylogenetic analysis and has involved the construction of a new enhanced topological model of rhomboid structure. In addition, the inventors have undertaken an in-depth biological study of a new member of the rhomboid family, RHBDL4. The invention is based upon the numerous insights derived from these rigorous parallel approaches.

One of the key findings to emerge from the analysis carried out is that RHBDL4 is in fact identified as a rhomboid protease. For numerous reasons which are explained in detail below, this finding is in contrast to the view currently held in the art. In addition to this, the RHBDL4 enzyme activity has been studied in considerable detail. This has led to significant insights into rhomboid protease activity. One example of these findings is the importance of orientation in the membrane to the cleavage of rhomboid substrates. Moreover, on a functional level, it has been demonstrated that each of the cleavage products of a rhomboid protease intramembrane cleavage event leaves the membrane.

In addition to these advances in understanding the mechanisms of rhomboid protease action, it has been clearly demonstrated that RHBDL4 is in fact restricted to the endoplasmic reticulum, and is therefore a secretase protease. This is in stark contrast to the prior art sequence based predictions regarding its location and activity. Lastly, and possibly of greatest biological significance, is the fact that RHBDL4 has been shown to mediate transactivation of the epidermal growth factor receptor (EGFR) by G-protein coupled receptors (GPCR's). EGFR transactivation has been clearly associated with a number of different diseases. Therefore, it can be appreciated that the invention is extremely significant both in the scientific and medical industries.

The present invention is based upon these surprising findings.

Thus, in one aspect the invention provides a method of inducing epidermal growth factor receptor (EGFR) transactivation in a system, said method comprising increasing RHBDL4 activity in said system.

Increasing RHBDL4 activity may refer to introduction or elevation of RHBDL4, or to activation of existing RHBDL4. Introduction may be by overexpression for example by introduction of a nucleic acid capable of directing expression of RHBDL4 polypeptide. Activation may be direct or indirect, for example by application of an activator of PKC which in turn leads to activation of RHBDL4.

Suitably said RHBDL4 activity induces shedding of pro-TGFalpha.

In another aspect, the invention provides a method of activating RHBDL4 in a system comprising activating protein kinase C (PKC) in said system. The activation of PKC may be by any suitable means known in the art such as addition of phorbol ester or related activator of PKC.

In another aspect, the invention provides a method of identifying a modulator of RHBDL4, said method comprising

(i) providing a first and second sample of cells
(ii) contacting said first sample of cells with a candidate modulator of RHBDL4
(iii) measuring epidermal growth factor receptor (EGFR) transactivation in said first and second samples of cells,
wherein a difference between the transactivation measured in said first and second samples of cells identifies said candidate modulator of RHBDL4 as a modulator of RHBDL4.

Clearly the cells must be chosen appropriately for the assay being carried out. Suitable cells comprise RHBDL4 and comprise a suitable transactivatable receptor such as a member of the HER receptor tyrosine kinase family such as the ErbB family of receptors, a subfamily of four related receptor tyrosine kinases: EGFR (ErbB-1), HER2/c-neu (ErbB-2), Her 3 (ErbB-3) and Her 4 (ErbB-4). Suitably the transactivatable receptor is EGFR (for convenience EGFR is typically referred to as the exemplary transactivatable receptor herein) for which transactivation can be assayed. The person skilled in the art will appreciate that the EGFR receptor itself can comprise different individual variants due to homo- or hetero-dimerisation at the cell surface. Exemplary cells and transactivatable receptors are noted in the examples section.

Advantageously an increase in transactivation in said first sample of cells relative to said second sample of cells identifies said modulator as a candidate activator of RHBDL4.

Advantageously a decrease in transactivation in said first sample of cells relative to said second sample of cells identifies said modulator as a candidate inhibitor of RHBDL4.

Suitably said transactivation is measured by assessing the level of BB94-insensitive release of EGFR ligand from said cells. Suitably said EGFR ligand is derived from higher molecular weight forms of TGFalpha comprising the entire ectodomain of TGFalpha that is post-translationally modified. As is well known to a person skilled in the art, the molecular weight may vary according to the degree of post translational modification. The important factor is to assess which molecular weight corresponds with the cleaved form(s). Suitably said EGFR ligand is the form of TGFalpha having an apparent molecular weight of 30 kDa or 37 kDa, suitably 37 kDa. Suitably said form of TGFalpha is detected via an amino acid sequence tag. Detection may suitably be by antibody against the TGFalpha domain.

Suitably said transactivation is stimulated via stimulation of a G-protein coupled receptor (GPCR). Suitably said GPCR is the gastrin releasing peptide receptor (GRPR) or the bombesin receptor, suitably the gastrin releasing peptide receptor. Said GPCR(s) may be present naturally on the cell(s) being assayed, or may be introduced for example by transduction such as transfection of a nucleic acid capable of directing the expression of same. Stimulation of said GPCR(s) may be by addition of appropriate ligand for said GPCR(s), such as bombesin for the bombesin receptor, or may be by addition of other moiety known to stimulate said receptor(s) such as stimulatory antibody or fragment thereof. Stimulation with insulin-like growth factor is an alternative to stimulation via GPCR in some embodiments.

Advantageously the transactivation assays disclosed herein are used in combination with a direct assessment of the effect of any modulator(s) on RHBDL4 activity itself. Suitably the RHBDL4 activity assessed in such embodiments is RHBDL4 protease activity. This may be measured by any suitable means such as those disclosed or described herein. The advantage of these combination assays, which may be conducted in either order or preferably in parallel (transactivation assay suitably being carried out in cells and direct RHBDL4 activity assay suitably being carried out in vitro e.g. using purified membranes or more suitably RHBDL4 protein), is that two indications are provided as to how the effect is being mediated. If transactivation is occurring, by also assaying the effect of the candidate modulator on RHBDL4 directly, then it is immediately validated as a RHBDL4 modulator (effectively reducing or eliminating the possibility that the transactivation is occurring via action on a non-RHBDL4 signalling component).

Thus it will be understood that the in vitro assays of RHBDL4 activity are specifically embraced in combination with the transactivation assays of RHBDL4 activity in preferred embodiments of the invention. They are described separately purely to aid understanding and reflect the modular nature of these combination embodiments.

Thus the invention provides a method as described above, further comprising the step of assaying the effect of said modulator on RHBDL4 protease activity. Suitably said RHBDL4 protease activity is determined as described below.

In another aspect, the invention provides use of a siRNA against RHBDL4 in the manufacture of a medicament for a disease associated with EGFR transactivation. Such diseases are well known to a person skilled in the art and include cancer, kidney disease or cardiovascular disease. Suitably said cancer is breast cancer.

Suitably said siRNA comprises the sequence of at least one of SEQ ID NO:1, SEQ ID NO:2 or SEQ ID NO:3.

In another aspect, the invention provides a method of treating cancer, kidney disease or cardiovascular disease comprising administering to a subject an effective amount of a siRNA wherein said siRNA comprises the sequence of at least one of SEQ ID NO:1, SEQ ID NO:2 or SEQ ID NO:3. Suitably said disease is breast cancer.

In a broad aspect, the invention relates to the use of recombinant or purified RHBDL4 as a protease, in particular as a rhomboid protease e.g. a protease for cleavage of ligands or pro-ligands. Suitably RHBDL4 is used as a secretase protease (see herein).

In another aspect, the invention provides use of recombinant or purified RHBDL4, or a catalytically active fragment thereof, as a protease. Use as a protease has its natural meaning in the art. RHBDL4 was not previously demonstrated to have protease activity. Indeed, this orthologue is considered to be missing from model organisms such as Drosophila in which rhomboids have previously been studied. Thus there has been no teaching of RHBDL4's protease function in the prior art. Thus it is a surprising benefit of the invention that use of RHBDL4 as a protease, such as a rhomboid protease, is now possible.

In another aspect, the invention provides use of recombinant or purified RHBDL4, or a catalytically active fragment thereof, as a rhomboid secretase protease. Use as a secretase protease means use in catalysing the release (secretion) of a polypeptide such as a TGFalpha polypeptide. This activity has been ascribed to RHBDL4 type proteases for the first time by the inventors. Indeed, the prior art mis-classified RHBDL4 as a PARL-type rhomboid, which is localised to the mitochondria, which teaches away from the present invention.

In another aspect, the invention provides use of recombinant or purified RHBDL4, or a catalytically active fragment thereof, in the cleavage of a polypeptide transmembrane domain.

In another aspect, the invention provides use of recombinant or purified RHBDL4, or a catalytically active fragment thereof, in the transactivation of EGFR.

In another aspect, the invention provides use of recombinant or purified RHBDL4, or a catalytically active fragment thereof, in the release of a substrate polypeptide from a membrane.

Suitably each of the cleavage products of said substrate polypeptide are released from the membrane. This is advantageous since prior art techniques have typically left one or more cleavage products in the membrane.

In another aspect, the invention provides a method of releasing a substrate polypeptide from a membrane, said method comprising contacting said substrate polypeptide with recombinant or purified RHBDL4, or a catalytically active fragment thereof. Suitably the polypeptide is cleaved by the RHBDL4 and each of the substrate polypeptide cleavage products is released from the membrane.

Suitably said substrate polypeptide is a TGFalpha polypeptide.

In another aspect, the invention provides a method of processing pro-TGFalpha, said method comprising contacting pro-TGFalpha with recombinant or purified RHBDL4 protein, or a catalytically active fragment thereof.

In another aspect, the invention provides a method of preparing active TGFalpha ligand comprising processing pro-TGFalpha as described above, and further comprising the step of contacting said processed TGFalpha with a metalloprotease.

Suitably said metalloprotease is an ADAM family metalloprotease. Suitably said metalloprotease is TACE.

In another aspect, the invention provides a method of identifying a modulator of RHBDL4 protease, said method comprising
(i) providing a first and second sample of RHBDL4 protease or a catalytically active fragment thereof;
(ii) contacting said first sample of RHBDL4 protease or catalytically active fragment thereof with a candidate modulator of RHBDL4; and
(iii) measuring cleavage of a RHBDL4 substrate by said first and second samples of RHBDL4 protease or catalytically active fragment thereof,
wherein a difference between the cleavage measured in said first and second samples of RHBDL4 protease or catalytically active fragment thereof identifies said candidate modulator of RHBDL4 as a modulator of RHBDL4.

Suitably said substrate comprises residues 224 to 272 of Drosophila Gurken. Suitably said cleavage is monitored by SDS-PAGE. Suitably a decrease in the protease activity determined in the first sample relative to the second sample indicates that said modulator is an inhibitor of RHBDL4 protease. Suitably an increase in the protease activity determined in the first sample relative to the second sample indicates that said modulator is an activator of RHBDL4 protease.

In another aspect, the invention provides a method of inhibiting transactivation of a HER tyrosine kinase family receptor, such as an ErbB family receptor n ErbB family receptor, in a system, said method comprising inhibiting RHBDL4 in said system.

Suitably said ErbB family receptor is the epidermal growth factor receptor (EGFR).

Suitably inhibiting RHBDL4 comprises introducing siRNA against RHBDL4 into said system. Suitably said siRNA comprises the sequence of at least one of SEQ ID NO:1, SEQ ID NO:2 or SEQ ID NO:3.

A system may be any system such as a biological system e.g. a cell based system or a cell or population of cells, or a cell free system or any reconstituted or synthetic system.

### DETAILED DESCRIPTION OF THE INVENTION

We describe for the first time a non-canonical pathway for TGFα secretion dependent on RHBDL4, an ER-resident rhomboid. We also describe a new mammalian rhomboid which mediates EGF receptor activation triggered by G-protein coupled receptor activation. We show that a newly discovered mammalian rhomboid gene RHBDL4 can efficiently release TGFα from cells. Moreover, we go on to provide evidence that EGFR transactivation by GPCRs, an increasingly important EGFR activation mechanism in disease, is mediated by rhomboid. This substantially revises current ideas about transactivation mechanisms. Our demonstration that RHBDL4 is a ER-resident protease is also significant, as the only other endoproteases in the ER are signal peptidase and SPP, and the ER is generally though to be a largely protease-free zone.

We disclose that a newly identified mammalian rhomboid, RHBDL4, can efficiently cleave human TGFalpha. We also demonstrate that RHBDL4 participates in transactivation of the EGFR by G-protein coupled receptors, evidencing a role for this rhomboid protease in pathogenic EGFR signaling. Unlike most proteases, RHBDL4 functions in the endoplasmic reticulum (ER) and we demonstrate that it triggers a non-canonical pathway for TGFalpha shedding in mammals.

In a broad aspect, the invention relates to RHBDL4 polypeptides and to nucleic acids encoding same. In particular, the invention relates to uses of, and methods involving, said polypeptides and/or nucleic acids as set out herein.

### EGFR Signaling

The epidermal growth factor receptor (EGFR) signaling pathway triggers diverse biological responses in development, and its hyperactivity is implicated in many human diseases alpha. EGFR ligands are typically synthesized as membrane tethered precursors and are only active upon proteolytic release from the cell membrane. In the case of TGF alpha, the best characterized mammalian EGFR ligand, the ADAM metalloprotease TACE is required for this activation. TGF alpha is trafficked to the plasma membrane by PDZ domain proteins, where TACE cleaves it just outside its transmembrane domain (TMD), releasing the active ligand. In Drosophila and C. elegans, the proteolytic activation of EGF-like ligands depends instead on rhomboid-family intramembrane serine proteases and, in Drosophila, these are known to be the cardinal regulators of developmental EGFR signaling. However, despite the widespread conservation of signalling pathways, EGFR ligand processing in mammals has been believed to be independent of rhomboid activity in the prior art.

TACE-independent shedding of TGFalpha, including an activity sensitive to the serine protease inhibitor DCI, induced us to pursue further the possibility of rhomboid involvement in mammalian EGFR control. To date, none of the mammalian rhomboids have any published activity against EGFR ligands. We disclose a new rhomboid, RHBDL4 and disclose its ability to cleave TGFalpha.

### Transactivation

We disclose herein the importance of RHBDL4 type rhomboids in EGFR transactivation. In contrast to the prior art which regards mammalian EGFR signalling as rhomboid independent, we describe how a mammalian rhomboid does indeed participate in EGFR control. We particularly highlight a role in pathogenic GPCR triggered transactivation of the EGFR.

EGFR stimulation in vivo can occur by ‘transactivation’, where GPCR signaling leads to the secondary release of EGFR ligands, which in turn activate the EGFR. (This transactivation is sometimes referred to as ‘crosstalk’.) Transactivation is also triggered by agents that stimulate protein kinase C (PKC), including phorbol esters like PMA. Transactivation has been implicated in cancer, as well as kidney and cardiovascular diseases.

### RHBDL4

References to ‘rhomboid’ or ‘rhomboid polypeptide’ should be construed accordingly with regard to the context. A ‘Rhomboid polypeptide’ as mentioned herein is suitably a RHBDL4 polypeptide or a RHBDL4 or secretase B family rhomboid. A RHBDL4 protease is a catalytically active RHBDL4 polypeptide, or fragment thereof. An exemplary RHBDL4 polypeptide is, or comprises, a vertebrate RHBDL4 such as a mammalian RHBDL4 polypeptide. Suitably the mammalian RHBDL4 polypeptide is mouse or human. Mouse RHBDL4 is advantageous for its relevance to the mouse as a key animal model and including numerous mouse cell lines and derivatives in common use in studies and screens in this area. Human RHBDL4 is particularly advantageous for the benefit of being most relevant to human systems and human disease, and as such may offer advantages in screening and testing embodiments. Mouse and human RHBDL4 are regarded as scientifically equivalent in that experiments presented which make use of mouse RHBDL4 are regarded as illustrative of human RHBDL4 and vice versa. Thus, evidence from mouse RHBDL4 is specifically applicable as evidence of human RHBDL4. Most suitably the RHBDL4 is human RHBDL4.

A fragment of a Rhomboid polypeptide such as RHBDL4 may consist of fewer residues than the full-length Rhomboid polypeptide. For example, a fragment of the RHBDL4 polypeptide may consist of less than 315 amino acid residues as described herein.

A Rhomboid/RHBDL4 polypeptide fragment consists of fewer amino acid residues than said full-length polypeptide. Such a fragment may consist of at least 255 amino acids, more preferably at least 300 amino acids. Such a fragment may consist of 305 amino acids or less, 300 amino acids or less, or 275 amino acids or less.

Such a fragment suitably comprises the conserved GxSx catalytic motif.

A suitable polypeptide fragment may comprise amino acid residues 5 to 210 of the full length human RHBDL4 sequence. For example, a polypeptide fragment may comprise residues 5 to 315 of the RHBDL4 protein and lack the N terminal cytoplasmic domain (tail) of the full length protein or may comprise residues 1 to 210 and lack the C terminal cytoplasmic domain of the full-length protein.

RHBDL4 consensus is derived from a ClustalW alignment of human, chimp, mouse, rat, xenopus and zebra fish RHBDL4.

A conserved motif GXSX (where X may be any amino acid residue) is frequently found around the active site serine residue, and a RHBDL4 polypeptide preferably comprises such a motif. In particular, the motif GFSG may be present.

In particular, suitably RHBDL4 polypeptides/secretase B type polypeptides and variants thereof described herein will possess one or more of the following motifs or residues:

### Motifs: RHBDL4-Specific Motifs/Consensus

Suitably a RHBDL4 polypeptide possesses one or more of the following characteristics (numbering refers to human RHBDL4 (Swiss-Prot accession No Q8TEB9); asterisked residues (X*) fit the rhomboid protease consensus; x stands for any amino acid; h stands for hydrophobic residue):

(i) A most pronounced characteristic for RHBDL4 orthologues is the basic six TMD topology (membrane integral portion from position 12 to 210) and a C-terminal putative globular domain (position 211 to 315). By contrast, Drosophila Rhomboid-1 has a N-terminal domain fused to the N-terminus of the basic rhomboid core and an additional TMD fused to the C-terminus leading to the characteristic 6+1 TMD topology of secretase A rhomboids.
(ii) WQR in the loop connecting TMD1 and TMD2 (WQR is found instead of the characteristic WR-motif found in the loop connecting TMD1 and TMD2 of non-RHBDL4 type rhomboid proteases).
(iii) phenylalanine in the first x-position of the GxSx active site motif

Suitably a RHBDL4 type rhomboid protease possesses two or more of the above characteristics, suitably all three of the above characteristics.

Moreover, suitably RHBDL4 type rhomboid proteases possess one or more of the following twelve motifs, suitably two or more, suitably three or more, suitably four or more, suitably five or more, suitably six or more, suitably seven or more, suitably eight or more, suitably nine or more, suitably ten or more, suitably eleven or more, suitably all twelve of the following characteristics:

(iv) RxRG (position 4 to 7; including putative RxR ER-retention signal)
(v) GLhLLhxQhFxhGhxNIPPVTLA (position 11 to 33)
(vi) FLxPxKPL (position 42 to 49)
(vii) DWxR*hLLSPhHH*xDDhH*LYFN* (position 64 to 84; suitably including variant of the characteristic WR-motif and the TMD2-signature (see above))
(viii) LWKGhxLE (position 89 to 96)
(ix) FSLxLxGhVY (position 111 to 119)
(x) CAVG*FS*GVLFxLKVxxNxYxPGG (position 139 to 161 including the catalytic S144)
(xi) ACWhELhhIH (position 175 to 184)
(xii) PGTSFhGH*xxGILVGLhYTxGPLK (position 188 to 211, including catalytic H195)
(xiii) SGY (position 240 to 242)
(xiv) YTxGhxEEEQ (position 264 to 273)
(xv) EEhRRxRhxRFD (position 302 to 213; suitably including putative RXR-type ER-retention signal)

### Residues:

A RHBDL4 fragment suitably comprises residues R67, G142, 5144 and H195, more suitably residues 5144 and H195, which are important for the catalytic activity of the protein and are highly conserved in the RHBDL4 secretase protease subfamily.

In particular, those shown as similar residues in FIG. 5 under the section ‘secretase B’ are especially suitable, most suitable are those shown as conserved.

A RHBDL4 polypeptide suitably includes HXXXXHXXXN in TMD2.

A RHBDL4 polypeptide suitably includes HXXGXXXG in TMD6.

A RHBDL4 polypeptide suitably includes GXSX in TMD4.

Amino acid residues of RHBDL4-type Rhomboid polypeptides are described in the present application with reference to their position in the RHBDL4 sequence, suitably the human RHBDL4 sequence for which the accession number is found below. It will be appreciated that the equivalent residues in other Rhomboid polypeptides may have a different position and number, because of differences in the amino acid sequence of each polypeptide. These differences may occur, for example, through variations in the length of the N terminal domain. Equivalent residues in Rhomboid polypeptides are easily recognisable by their overall sequence context and by their positions with respect to the Rhomboid TMDs.

A Rhomboid polypeptide may also comprise additional amino acid residues which are heterologous to the Rhomboid sequence. For example, a fragment as described above may be included as part of a fusion protein, e.g. including a binding portion for a different ligand.

A Rhomboid polypeptide suitable for use in accordance with the present invention may be a member of the RHBDL4 or secretase B family, most suitably a RHBDL4 type polypeptide, or a mutant, homologue, variant, derivative or allele thereof. A polypeptide which is a RHBDL4 type polypeptide or which is an amino acid sequence variant, allele, derivative or mutant thereof may comprise an amino acid sequence which shares greater than about 18% sequence identity with the sequence of human RHBDL4, greater than 25%, greater than about 35%, greater than about 40%, greater than about 45%, greater than about 55%, greater than about 65%, greater than about 70%, greater than about 80%, greater than about 90% or greater than about 95%. The sequence may share greater than about 30% similarity with human RHBDL4, greater than about 40% similarity, greater than about 50% similarity, greater than about 60% similarity, greater than about 70% similarity, greater than about 80% similarity or greater than about 90% similarity.

As will be apparent from the specification as a whole, RHBDL4 type rhomboids are identified on more criteria than pure sequence identity/similarity—preferably members of the RHBDL4 family share one or more other properties or characteristics as set out herein.

Preferably, an amino acid sequence variant, allele, derivative or mutant of a polypeptide of the RHBDL4 family retains RHBDL4 activity i.e. it proteolytically cleaves a TGFalpha substrate as described herein.

### Sequence Identity/Similarity

Sequence similarity and identity is commonly defined with reference to the algorithm GAP (Genetics Computer Group, Madison, W7). GAP uses the Needleman and Wunsch algorithm to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mot Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine. Particular amino acid sequence variants may differ from a known RHBDL4 polypeptide sequence as described herein by insertion, addition, substitution or deletion of 1 amino acid, 2, 3, 4, 5-10, 10-20 20-30, 30-50, or more than 50 amino acids.

Sequence comparison may be made over the full-length of the relevant sequence described herein, or may more preferably be over a contiguous sequence of about or greater than about 20, 25, 30, 33, 40, 50, 67, 133, 167, 200, 233, 267, 300, 310, or more amino acids or nucleotide triplets, compared with the relevant amino acid sequence or nucleotide sequence as the case may be.

### Substrates

A suitable RHBDL4 substrate may consist of or may comprise a transmembrane domain which includes a RHBDL4-cleavable motif which has an equivalent conformation, structure or three dimensional arrangement to that of the corresponding residues of the TGFalpha sequence (see FIG. 2).

As described above, the substrate is cleaved by the RHBDL4 polypeptide within the transmembrane domain.

Other suitable polypeptide substrates may comprise a transmembrane motif which, although lacking high sequence identity with the substrate region of TGFalpha, nevertheless possesses a motif having an equivalent structure to TGFalpha or other peptide which is cleaved by RHBDL4 polypeptide.

Suitable RHBDL4 substrates include:

Drosophila Spitz (Swiss-Prot accession Q01083)
Drosophila Gurken (Swiss-Prot accession P42287)
human pro-TGFalpha (Swiss-Prot accession P01135)
human pro-HB-EGF (Swiss-Prot accession Q99075)
human pro-Amphiregulin (Swiss-Prot accession P15514)
mouse pro-Betacellulin (Swiss-Prot accession Q05928)
mouse TGN46 (homologue of rat TGN38; Swiss-Prot accession Q62313).

Variants, derivatives or homologues of these may equally serve as substrates provided they retain the property of being cleavable by RHBDL4, which can be easily verified as taught herein.

Suitable negative controls i.e. moieties not cleaved by RHBDL4 include:

human pro-EGF (Swiss-Prot accession P01133)
human calnexin (Swiss-Prot accession P27824)
mouse Site 1 protease (SIP; Swiss-Prot accession Q9WTZ2)
mouse ADAM17/TACE (Swiss-Prot accession Q9Z0F8)
mouse thrombomodulin (Swiss-Prot accession P15306).

Regarding other mammalian such as human/mouse growth factors which may be candidate substrates, proEpiregulin and proEpigen may be tested and used as appropriate in the present invention.

For example, a suitable polypeptide substrate may include an amino acid sequence consisting of the transmembrane region of Drosophila Spitz polypeptide, Golgi protein TGN46 (TGN38), or chimaeric substrates comprising amino acid residues from two or more such individual substrates for example as set out in the examples section and in particular in FIG. 2 or a variant, allele, derivative, homologue, or mutant thereof.

It should be noted that in order to determine whether or not a candidate is indeed a substrate of RHBDL4, it can simply be tested for RHBDL4 cleavage following the techniques and guidance provided herein.

A variant, allele, derivative, homologue, or mutant may consist of a sequence having greater than about 50% sequence identity with the transmembrane region of the reference substrate polypeptide such as TGFalpha, greater than about 60%, greater than about 70%, greater than about 80%, greater than about 90%, or greater than about 95%. The sequence may share greater than about 70% similarity with the sequence of the transmembrane domain of the reference substrate polypeptide such as TGFalpha, greater than about 80% similarity, greater than about 90% similarity or greater than about 95% similarity. Preferably, such a variant, allele, derivative, homologue, or mutant comprises residues of the RHBDL4 cleavable substrates such as TGFalpha substrate as shown in FIG. 2, or residues with an equivalent secondary structure or conformation.

Detection of substrates and/or cleavage is typically by assessing the molecular weight pre- and post-treatment with protease. Suitable substrates may advantageously comprise further means for detection. This may comprise radioactive label, or may comprise further amino acid sequence joined (e.g. fused) to the substrate to facilitate for example detection by antibody or collection/capture of substrate, or cleaved elements thereof, such as His8 tag or other amino acid sequence tag known in the art, or other detectable label such as fluorescent label.

### RHBDL4 Assays

RHBDL4 may be assayed in vitro. Suitably the mammalian protein is assayed. Suitably the human protein is assayed.

Firstly, a protein is over expressed in a suitable host cell. This may be any organism. Suitably a host cell may be E. coli, which is advantageously easy to manipulate in vitro. More suitably, the host cell may be eukaryotic. A suitable host cell may be a yeast host cell such as S. pombe or S. cerevisiae. Mammalian cells are particularly suitable, such as mammalian tissue culture cells, for example HEK293 T-cells.

The over expressed RHBDL4 protein is then solubilised.

The solubilised RHBDL4 protein may then be purified. Suitably, purification may be by affinity purification. RHBDL4 activity may be assayed with suitably purified material or in a crude membrane fraction from cells overexpressing the protein.

The RHDBL4 protein such as recombinant RHBDL4 protein (e.g. purified or membrane fraction) is then added to the substrate polypeptide. The substrate polypeptide may suitably be chosen from one or more of those disclosed examples, such as a TGFalpha polypeptide.

Cleavage of the polypeptide by the RHBDL4 protein is then assessed.

A particularly suitable technique for the assay of RHBDL4 activity as outlined above may be based on the method disclosed in Lemberg et al (EMBO 2005 Volume 24 pages 464-472). In particular, the materials and methods section of this publication describes in detail how rhomboid assays may be carried out. Clearly, RHBDL4 is substituted for RHBDL2 in using the guidance presented in Lemberg et al, which is well within the abilities of the person skilled in the art. Lemberg et al is incorporated herein by reference in its entirety.

In more detail, the steps of in vitro RHBDL4 assays may be performed as follows:

To produce recombinant RHBDL4, suitably a RHBDL4-purification tag fusion protein is expressed and affinity purified. For example, C-terminally His6-tagged RHBDL4 may be expressed in E. coli BL21-Gold(DE3) cells harbouring the expression vector and the extra plasmid pRARE2 (Novagen) as described for human RHBDL2 (Lemberg, 2005, EMBO vol 24 pp 464-472). Alternatively, RHBDL4 may be expressed in yeast, insect cells or mammalian tissue culture cells. In order to get a fast and efficient purification of correctly folded membrane proteins from yeast, a fusion protein with an oxalate decarboxylase domain, which is naturally biotinylated in yeast, may be used. Suitably this may be purified using avidin agarose affinity chromatography for a one step purification (Pouny et al. 1998 Biochemistry 37: 15713-15719).

After the protein expression, cells are disrupted and membranes containing the recombinant RHBDL4 may be harvested by centrifugation as has been described (Lemberg 2005 above). Alternatively cells may be broken by standard methods including French press or sonication or enzymatic cell lysis.

Subsequently the recombinant protein may be solubilised with the detergent Triton X-100. The activity may be assayed directly in this solubilised membrane fraction or may be affinity purified using Ni2+-NTA Superflow gravity column as has been described for the bacterial homologues GlpG and YqgP (Lemberg, 2005 above). Alternatively other detergents such as DDM, NP-40 C12E8, or combinations thereof, may be used.

To conduct the cleavage assay, radiolabelled substrate comprising or consisting of the substrate TMD may be generated by cell-free in vitro translation using wheat germ extract and [35S]methionine as has been described (Lemberg and Martoglio, 2003 Anal Biochem. vol 319 pp 327-31). One such suitable substrate corresponds to an N-terminal methionine plus residues 224 to 272 of Drosophila Gurken. Other substrate TMDs such as human TGFalpha, human HB-EGF, Drosophila Spitz may be used instead.

As an alternative to such in vitro translated peptides, recombinant substrates or chemically synthesized peptides may be used; e.g. substrates expressed in E. coli and purified from detergent solubilised membranes as has been described (Stevenson, 2007, PNAS 104:1003-1008).

For the cleavage assay, typically 1-4 μl in vitro translation mix or 50-200 μg/ml recombinant substrate are added to a 40 μl-reaction containing recombinant RHBDL4 or a crude membrane preparation comprising RHBDL4 (suitably approximately 1-5 pg of RHBDL4 are present) in 50 mM HEPES/NaOH, pH 7.4, 10% glycerol and 50 mM EDTA. Samples are incubated at 30° C. and subsequently the cleavage reaction is analyzed (e.g. by SDS-PAGE as described, Lemberg, 2005). Alternatively HPLC, or fluorescence based detection of chemically modified substrates may be used.

Clearly it is well within the abilities of the person skilled in the art to optimise conditions to suit their particular need or application/format.

### Cell Based Assays

RHBDL4 protease activity may also be assessed in a cell based system. In this embodiment, the method disclosed for RHBDL2 in WO 2005/069011 is suitably used. It should be noted that RHBDL2 is in the late secretory pathway. This cellular compartment tends to include a lot of ADAM protease activity. This activity can produce extra cleavage events and therefore provide substantial background in the assay. There are numerous ways in which this may be overcome. Firstly, BB94 inhibitor may be used in order to block unwanted protease activity. Alternatively, detection of a specific epitope in the juxtamembrane position may be employed in the assay. Cleavage by TACE proteases releases the epitope, whereas cleavage by rhomboid proteases leaves the epitope, thereby allowing easy distinction between TACE and rhomboid protease action. However, it is an advantage of the present invention that RHBDL4 activity is located in the endoplasmic reticulum (ER). It is beneficial that the interfering proteases discussed above are not typically present in the ER. Therefore, the assay disclosed in WO 2005/069011 may be adapted to omit the use of BB94 inhibitor, and/or to omit the use of the epitope in the juxtamembrane position. Furthermore, techniques used to detain the rhomboids in the endoplasmic reticulum to avoid the types of problems outlined above are also not necessary for RHBDL4, since advantageously, this protein is naturally restricted to endoplasmic reticulum anyway. Thus, cell based assays of RHBDL4 activity disclosed herein are advantageously cleaner and easier than prior art based methods. WO 2005/069011 is incorporated herein in its entirety.

A most suitable method for assay of RHBDL4 activity is the transactivation assay such as the EGFR transactivation assay. The benefits of using this assay are that it provides a genuine biological readout for RHBDL4 activity such as endogenous RHBDL4 activity by G-protein coupled receptors, a documented function of RHBDL4. The methods for measuring EGFR transactivation are well known to those skilled in the art and have been published. It should be noted that this pathway relies at least partially on the activity of ADAM metalloproteases, which may cause background cleavage of transactivation substrates. As described herein, RHBDL4 activity can be assayed in the presence of BB-94 metalloprotease inhibitor. Moreover, the RHBDL4 contribution to EGFR transactivation can also be assessed by genetic techniques such as siRNA knockdown.

It will be clear to the skilled reader and from the guidance given herein that the invention finds application in identification of agents (such as compounds, biological entities such as genes, or particular treatments or conditions) which affect rhomboid function. Thus, each of the assays described herein may advantageously be applied to screening, for example by performing assays in duplicate with one treatment exposed to the particular compound or other entity under test, and the other treatment not so exposed, and by comparison of the results from the duplicated treatments. Differences between the treatments indicate effect(s) of the test compound or entity. Directional differences (e.g. increase or decrease of activity) provide further information useful to the operator. Various exemplary embodiments are described herein, such as identification of candidate drugs affecting RHBDL4 activity such as protease and/or transactivation activity. Other embodiments will be apparent to the skilled reader.

### Agent

As used herein, the term “agent” or “candidate modulator” may be a single entity or it may be a combination of entities. Preferably, the agent modulates the activity of RHBDL4.

Thus, the agent may be an antagonist or an agonist of RHBDL4. Preferably, the agent is an antagonist of RHBDL4.

The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule. The agent may even be an antibody. The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the agent will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agent comprises at least the one of said cyclic groups linked to another hydrocarbyl group.

The agent may contain halo groups, for example, fluoro, chloro, bromo or iodo groups.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, (1977) J. Pharm. Sci. 66, 1-19.

The agent of the present invention may be capable of displaying other therapeutic properties.

The agent may be used in combination with one or more other pharmaceutically active agents.

### Host Cells

Vectors/polynucleotides encoding RHBDL4 polypeptides of the invention may introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

### Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption. In particular it is advantageous to solubilise the RHBDL4 polypeptides of the invention as is well known to those skilled in the art.

### Administration

Proteins of the invention, and/or substances identified or identifiable by the assay methods of the invention, may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptides of the invention may be administered directly as a naked nucleic acid construct, preferably further comprising flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example Lipofectam™ and Transfectam™). Typically, nucleic acid constructs are mixed with the transfection agent to produce a composition.

Preferably the polynucleotide or polypeptide of the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosages described are intended only as a guide since a skilled practitioner will be able to determine readily the optimum route of administration and dosage for any particular patient and condition.

### INDUSTRIAL APPLICATION

In addition to the applications apparent from the specification as a whole, the invention finds particular application and utility in several fields including cancer, growth factor signalling, membrane trafficking, intramembrane proteases, development and cell biology. The invention may be applied to industrial studies, screens for chemical entities and to manufacture of medicaments for treatment of disease. Furthermore, the disclosure of novel function for RHBDL4 is useful in the industry.

### Further Applications

In addition to providing methods for production of active TGFalpha ligand by use of recombinant or purified RHBDL4 enzymes, the present invention also embraces methods for production of active TGFalpha ligand comprising activating RHBDL4, and optionally activating one or more metalloproteases.

It is desirable to suppress RHBDL4 activity. This may be accomplished by down regulating the protein, by inhibiting its activity, by suppressing or down regulating its expression, or by any other suitable means known in the art. Diseases in this field which have been characterised to date are associated with too much EGFR signal, too much ligand release, too much EGFR receptor, or other excess of signal. As disclosed herein, RHBDL4 is intimately involved in the biological processing and/or release of ligand such as TGFalpha. Therefore, by down regulating RHBDL4, the excessive activity associated with disease is advantageously suppressed or reduced. A suitable technique for down regulating RHBDL4 is the use of short interfering RNA (siRNA) to target RHBDL4.

In some embodiments, it may be advantageous to combine down regulation of RHBDL4 with down regulation of serine proteases. For example, a serine protease inhibitor may be combined with down regulation of RHBDL4. The advantage of this embodiment is that serine proteases (such as metallo proteases) are required to produce active ligand from the RHBDL4 process pro-protein. Therefore, by also targeting the downstream proteases involved in producing the active ligand, an additive or even synergistic effect may be achieved.

It is a further aspect of the invention to formulate the modulators of RHBDL4 identified according to the present invention for use in medicine. Thus, preferably such methods used to identify modulators of RHBDL4, particularly inhibitors of RHBDL4, further comprise the step of formulating said candidate modulator or agent into a pharmaceutically acceptable form. Pharmaceutically-acceptable salts are well known to those skilled in the art, and for example include those mentioned by Berge et al, (1977) J. Pharm. Sci., 66, 1-19. Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent may be readily prepared by mixing together solutions of the agent and the desired acid or base, as appropriate. The salt may precipitate from solution and be collected by filtration or may be recovered by evaporation of the solvent.

The agent may exist in polymorphic form.

The agent may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Medicinal uses of RHBDL4 inhibition or down-regulation (i.e. uses of inhibitors or down-regulators) include those noted herein as well as application in human carcinomas such as breast cancer (ligand=estrogen; GPCR is GPR30); colon cancer (ligand=carbachol); ovarian carcinoma (ligand=interleukin8). Moreover, it is useful in Helicobacter pylori induced inflammatory processes leading to gastric carcinogenesis; kidney disease (ligand=angiotensin II); cardiovascular disease (ligand=HB-EGF—see examples); lung cancer (ligands comprised by cigarette smoke) and Staphylococcus aureus infection.

The medical uses are particularly suitable for application to disorders of EGFR signalling, including when the EGFR ligand is EGF or HB-EGF or related entity.

### BRIEF DESCRIPTION OF THE FIGURES

FIG. 1. TGFalpha is cleaved by RHBDL4

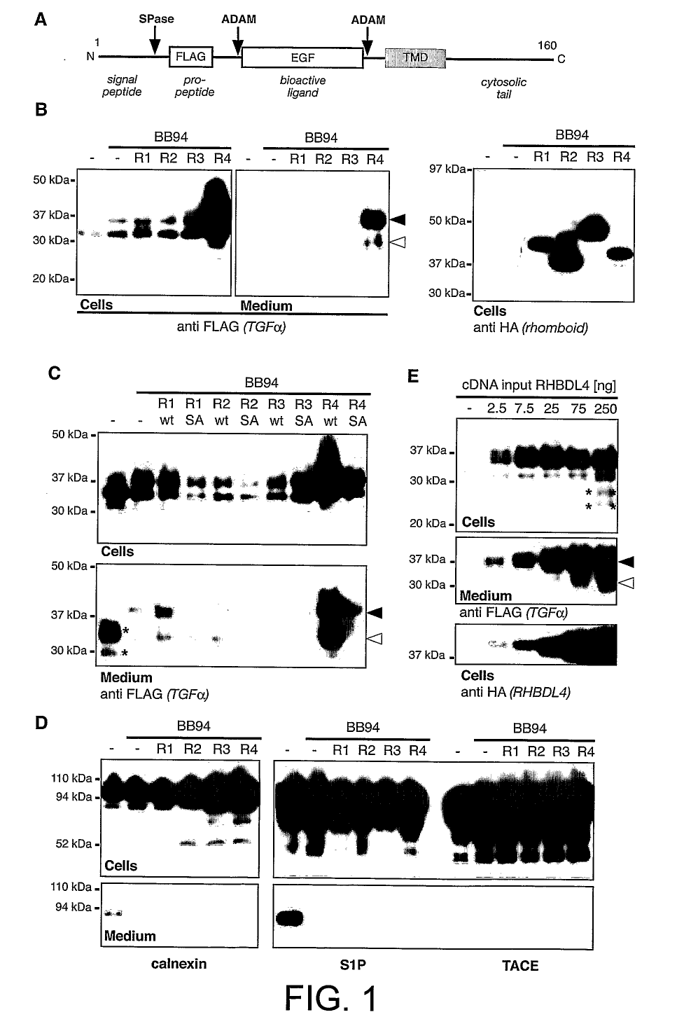

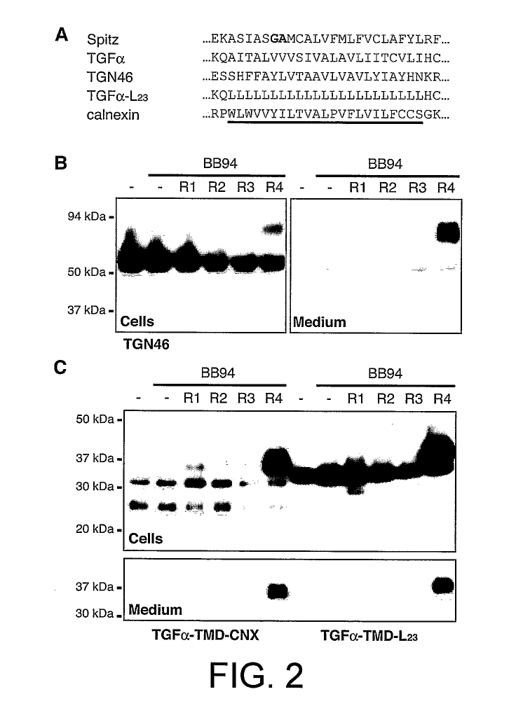

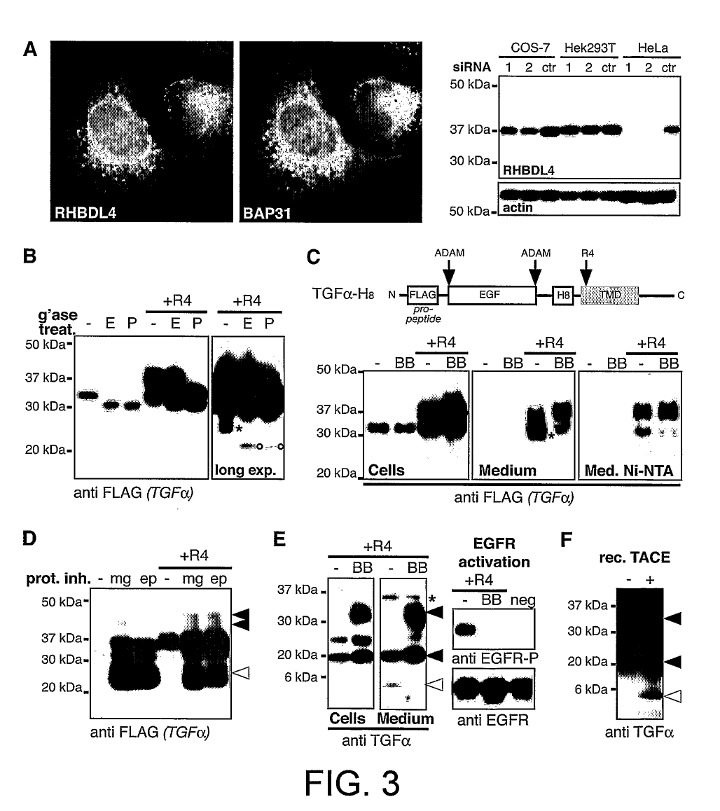

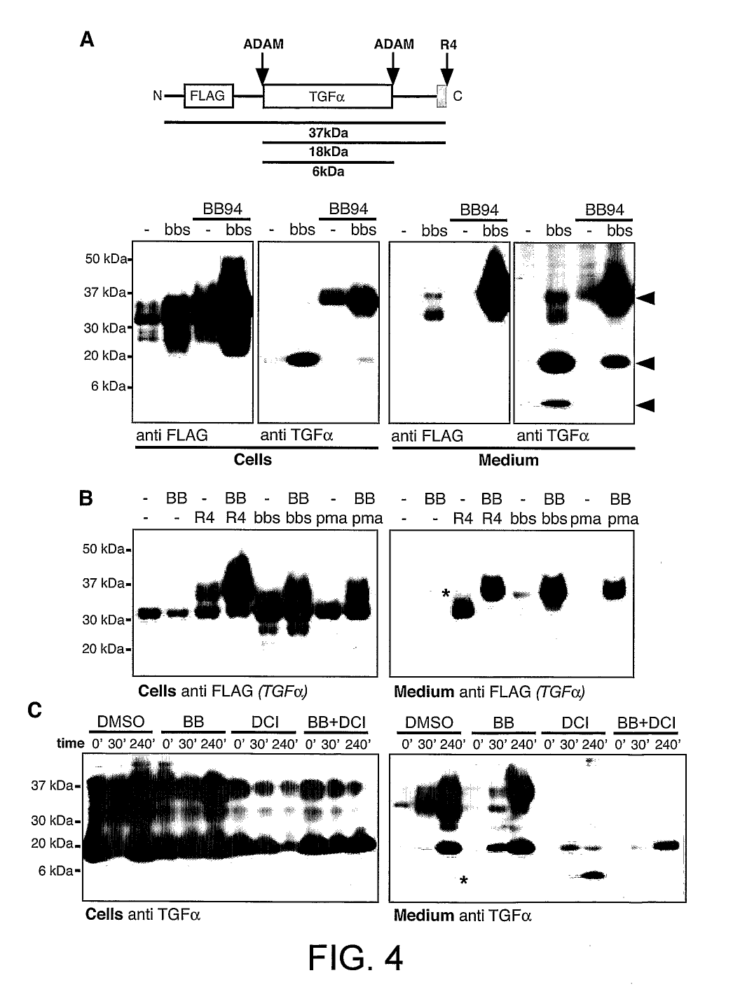

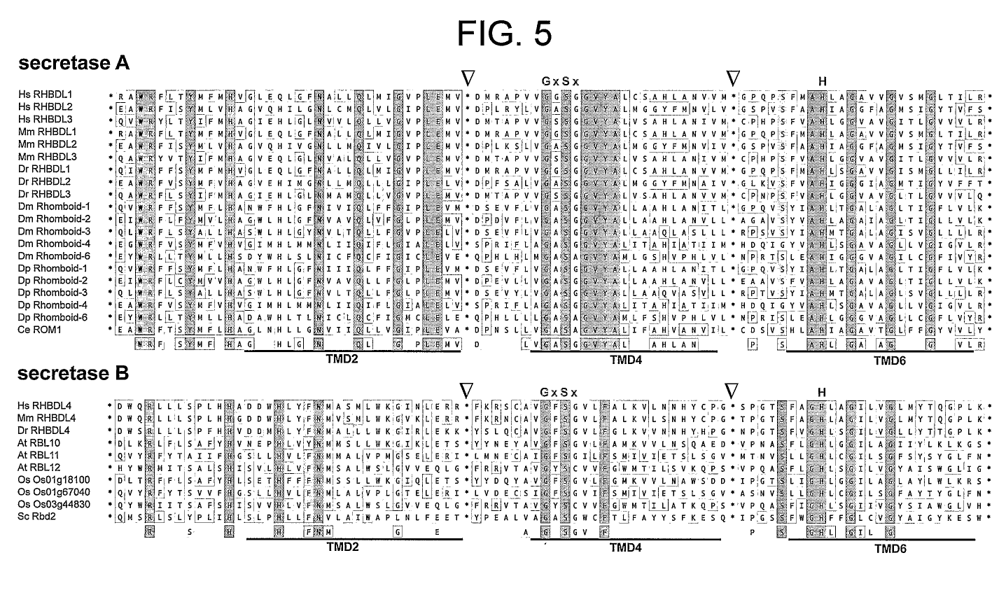

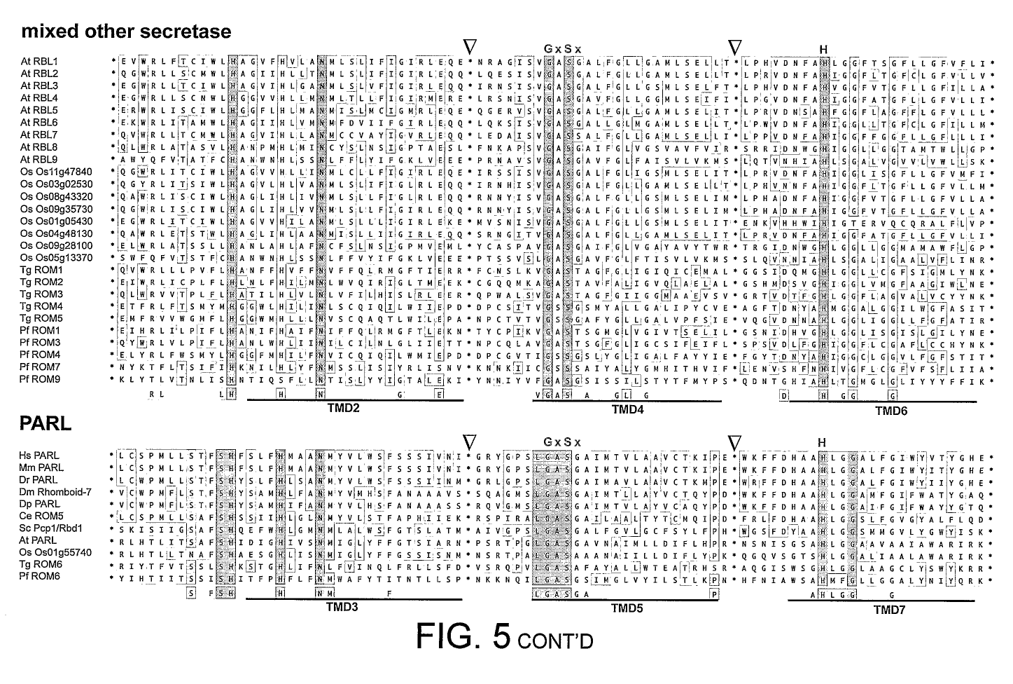

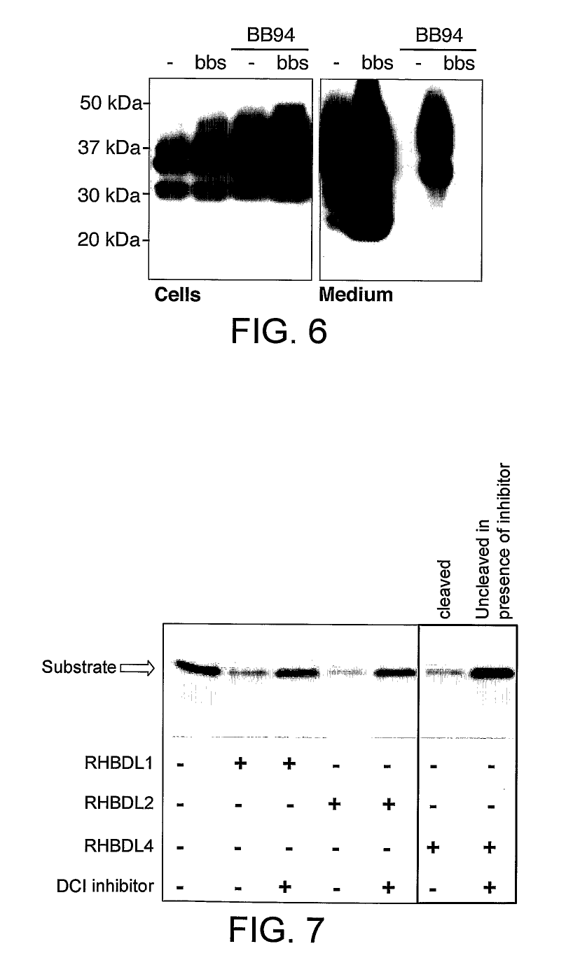

(A) Schematic representation of pre-pro-TGFalpha. Position of the FLAG-tag is indicated. (B) Western blot showing that mouse RHBDL4 (R4) but not the other mouse rhomboids (R1, R2 and R3) triggered the generation and secretion of a 37 kDa form, and traces of a 30 kDa form, of TGFalpha. filled and open triangle respectively). Pro-TGFalpha (34 kDa) was detected at low levels in the absence of RHBDL4, so the blot of cell extracts is overexposed compared to the blot of medium. Rhomboid expression was detected by the HA₃-tag (right panel). The assay (except lane 1) was performed in the presence of 10 μM BB94 to inhibit unspecific shedding by ADAM proteases. (C) Increasing sensitivity of the cleavage assay (by use of a FLAG₆-tag, which adds an extra 3 kDa MW) showed endogenous shedding of pro-TGFalpha. Generation of the higher MW forms (filled and open triangles, see FIG. 1B) was insensitive to BB94 (20 μM). In contrast, trimming to smaller intermediates (asterisks) and species lacking the pro-peptide (not detected by anti-FLAG) was blocked by BB94 (see also FIG. 4A). Note that overexpression of RHBDL1 caused a minor increase of secreted 37 kDa product. (D) Calnexin, S1P and TACE are not cleaved by RHBDL4. (E) RHBDL4 cleaves pro-TGFalpha in sub-stoichiometric amounts. Asterisks label intracellular low MW cleavage products; triangles indicate the secreted higher MW forms (as in FIG. 1A). The cDNA input for pro-TGFalpha was kept constant (250 ng).

FIG. 2. RHBDL4 is more aggressive than other rhomboids.

(A) RHBDL4-catalysed processing does not require classical rhomboid substrate features. TMD-sequence of Drosophila Rhomboid-1 substrate Spitz, TGFalpha, TGN46, TGFalpha-TMD-L₂₃ and the negative control calnexin. The predicted membrane-spanning region is underlined and the GA-motif necessary for Spitz processing is highlighted (13). Note that RHBDL4 cleaves Spitz. (B) and (C) Mouse RHBDL4, but not other rhomboids, cleaved TGN46 (B) and the chimeric molecules TGFalpha-TMD-CNX and TGFalpha-TMD-L₂₃ (C).

FIG. 3. RHBDL4 is an ER-localized intramembrane protease.

(A) Immunofluorescence analysis of untransfected COS-7 cells shows RHBDL4 co-localizes with the ER protein BAP31. Western analysis of siRNA-treated cells (two independent oligos 1, 2 and ctr for control) showed that the RHBDL4 antibody was specific. (B) RHBDL4 cleaves pro-TGFalpha in the ER, as demonstrated by the sensitivity of the lower MW product (asterisk) generated by RHBDL4 to EndoH (H) (open circle for deglycosylated form). Similarly, unprocessed pro-TGFalpha (34 kDa) was sensitive to EndoH, but the 37 kDa form seen after RHBDL4 overexpression was only deglycosylated by PNGaseF (P), indicating that it had been modified in the Golgi. (C)RHBDL4 cleaves near the luminal end of the TMD. Upper panel: schematic of the construct. Lower panel: capture by Ni-NTA of three secreted species of the TGFalpha ectodomain (varying in post-translational modification); the 28 kDa form generated by BB94-sensitive trimming (asterisk) was not captured. BB stands for BB94. (D) Treatment with proteasome inhibitors MG132 (mg; 5 μM) and epoxomicin (ep, 2 μM) led to unglycosylated TGFalpha (open triangle) and several higher MW forms (filled triangles) characteristic of cytosolic accumulation and polyubiquitination of proteins dislocated from the ER (E) RHBDL4-processed TGFalpha cannot activate the EGFR efficiently. Left panel: Western analysis of untagged TGFalpha showing secretion of the higher MW species and a previously not recognized 18 kDa form that lacks the pro-peptide (but which is further modified and not bioactive). In the absence of BB94 the higher MW species (filled triangles) are converted into the bioactive 6 kDa form of TGFalpha (open triangle). The asterisk indicates a background band. Right panel: Western analysis of A431 cells detected phosphorylated EGFR only upon incubation with conditioned medium containing the mature 6 kDa form of TGFalpha. (F) Recombinant TACE cleaved the post-translationally modified higher MW forms of TGFalpha. (filled triangles), generating the mature 6 kDa form (open triangle).

FIG. 4. EGFR transactivation mediated by RHBDL4.

(A) Treatment with bombesin (bbs) of COS-7 cells overexpressing the bombesin receptor stimulated TGFalpha secretion. The 37 kDa form was processed in a BB94-sensitive way to form the 6 kDa secreted bioactive ligand via a number of intermediates (triangles indicated major forms; see upper panel for schematic representation; note that the 37 kDa and 181kDa forms are post-translationally modified). (B) TGFalpha secreted by endogenous BB94-insensitive activity (asterisk) mimicked shedding induced by PMA, bombesin (bbs; in the presence of overexpressed receptor), and overexpressed RHBDL4. BB94-sensitive (i.e. metalloprotease dependent) trimming was also enhanced by PMA and bombesin. BB stands for 20 μM BB94. (C) Time course after PMA induction of HEK293T cells overexpressing pro-TGFalpha was performed in presence of BB94 (BB, 20 μM), DCI (100 μM) or both. The release of the 37 kDa form of TGFalpha was inhibited by DCI, however the canonical pathway leading to the direct release of the 6 kDa form was not (minor band indicated by asterisk; enhanced by DCI treatment). BB94 has a converse effect: the 6 kDa form was inhibited but the 37 kDa form was not. The 181 kDa band that is apparently insensitive to DCI and BB94 represents secreted TGFalpha processed before the beginning of the time course.

FIG. 5 shows RHBDL4 alignment and consensus.

FIG. 6 shows bombesin induced BB94-insensitive activity; the experiment was performed analagous to FIG. 4A but with N-terminal FLAG3-tagged HB-EGF as explained below.

FIG. 7 shows an annotated photograph of the results of an in vitro activity assay with recombinant mouse RHBDL4, i.e. an in vitro cleavage assay with RHBDLs.

The invention is now described by way of example. These examples are intended to be illustrative, and are not intended to limit the appended claims.

### EXAMPLES

### Example 1

### TGFalpha Processing

TGFalpha processing intermediates are complex, with, in addition to the cleavage that releases the mature growth factor, proteolytic removal of the N-terminal pre-pro-domain, and a variety of modifications (FIG. 1A). We found that RHBDL4 cleaves pro-TGFalpha efficiently in COS-7 cells (FIG. 1B) as well as in HeLa and HEK293T cells (see below). Cleavage is insensitive to the potent metalloprotease inhibitor BB94, and depends on the rhomboid catalytic serine (FIGS. 1B and C). By increasing the sensitivity of the assay, a low level of BB94-insensitive endogenous activity is also observed (FIG. 1C). Both this endogenous activity and RHBDL4 overexpression caused the secretion of a 37 kDa form of TGFalpha; significantly, this coincides with a form of TGFalpha generated in vivo in response to transactivation by G-protein coupled receptors (GPCRs). When ADAMs were not inhibited by BB94, the higher MW forms of TGFalpha were no longer detected, and trimming to smaller species was observed (FIG. 1C). We interpret this to be caused by ADAM-catalyzed trimming, which is consistent with observed in vitro processing of both cleavage sites flanking the bioactive TGFalpha by TACE (FIG. 1A).

As well as triggering cleavage, RHBDL4 led to substantially increased levels of intracellular TGFalpha; this was caused by protection from degradation and is analyzed below. RHBDL4 did not cleave other type I membrane proteins including calnexin, S1P protease and TACE (FIG. 1D), which are localized in the ER, the Golgi apparatus and the plasma membrane respectively, implying that, like other rhomboids, RHBDL4 has substrate specificity. As expected for an enzyme, cleavage of TGFalpha requires sub-stoichiometric amounts of RHBDL4 (FIG. 1E). Modification later in the secretory pathway caused most of the RHBDL4-cleaved TGFalpha to run at a higher MW than pro-TGFalpha (see below). In the presence of high levels of enzyme, however, two smaller bands, the primary cleavage products, were visible (FIG. 1E). In summary, we disclose that RHBDL4 is a novel pro-TGFalpha processing enzyme.

A key determinant of rhomboid substrates is the presence of helix destabilizing residues in the TMD. The TGFalpha TMD has no obvious motifs of this kind so we investigated this further (FIG. 2A). RHBDL4 appears more promiscuous than other rhomboids. For example, it cleaved the Golgi protein TGN46 (FIG. 2B) (mouse orthologue of rat TGN38), which lacks helical disrupting residues and is uncleaved by other rhomboids. Despite not cleaving calnexin (see above), a chimeric protein comprising TGFalpha with the TMD of calnexin, was cleaved (FIG. 2C). It was also active against a molecule in which the TMD of TGFalpha was replaced with 23 leucines, predicted to have a very high helical propensity (FIG. 2C). Our evidence therefore shows that although RHBDL4 shows substrate specificity, it cleaves TMDs without typical rhomboid determinants; it also implies that regions outside the substrate TMD can influence cleavage, as is the case for RHBDL2.

### RHBDL4 Processing

To investigate how RHBDL4 cleavage relates to TACE processing, we raised an antibody against RHBDL4 and found that the endogenous protein colocalises with an ER marker, BAP31 (FIG. 3A). Consistent with this, RHBDL4 has cytoplasmic RxR motifs in its N- and C-terminal tails that are predicted to be ER retention signals. Therefore RHBDL4 is expected to be active in the ER, earlier in the secretory pathway than TACE, which is inactive until it reaches the trans-Golgi network. Such compartmentalization is consistent with the different modified forms of TGFalpha we detect. In fact, it has been reported previously that the majority of pro-TGFalpha is retained in the ER where it is not susceptible to TACE cleavage. Using the deglycosylating enzymes EndoH and PNGaseF to distinguish ER from Golgi forms of processed TGFalpha, we found that the minor bands around 25 and 22 kDa (as seen in FIG. 1E) are located in the ER, whereas the higher MW bands represent modifications that occur later in the secretory pathway (FIG. 3B). Together with the ER-localization of RHBDL4, this implies that the smaller forms are the initial RHBDL4 cleavage products and confirms that this processing occurs in the ER.

### Cleavage Site

Rhomboids cleave within TMDs, whereas TACE and other metalloproteases catalyze juxtamembrane cleavage. To examine where TGFalpha is cleaved by RHBDL4, we incorporated a His₈-tag between the juxtamembrane TACE cleavage site and the TMD (FIG. 3C). RHBDL4 triggered the expected BB94-insensitive TGFalpha release in HEK293T cells and this is bound by Ni-NTA resin, which recognizes the His₈-tag. In the absence of BB94 we see a slightly smaller form of secreted TGFalpha that is not bound by Ni-NTA; this we assume to be a form in which the secreted ectodomain has been further trimmed by metalloproteases to remove the His₈-tag (similar trimming was noted in FIG. 1C). Together these results directly confirm that RHBDL4 induced cleavage occurs C-terminal to the His₈-tag, near the luminal end of the TMD, a hallmark of rhomboid proteolysis.

As noted above, RHBDL4 coexpression led to increase in intracellular TGFalpha. This dramatic increase depended on the catalytic serine (FIG. 1C), demonstrating that it was directly caused by rhomboid proteolytic activity. Using proteasome inhibitors, we found that pro-TGFalpha is highly susceptible to proteasomal degradation (FIG. 3D). This demonstrates that under steady state conditions the majority of newly synthesized pro-TGFalpha does not leave the ER but is degraded by ER associated degradation (ERAD). A proportion of this pro-TGFalpha escapes ERAD by being trafficked to the plasma membrane by PDZ domain proteins that interact with its cytoplasmic tail. Thus, we show that intramembrane cleavage of pro-TGFalpha by RHBDL4 in the ER provides an alternative route for TGFalpha secretion and escape from ERAD.

### TGFalpha Shedding

We investigate whether there is a biological distinction between TACE and RHBDL4 mediated TGFalpha shedding. We examined the activation of the EGFR by RHBDL4-processed extracellular TGFalpha, and found that, in contrast to TACE-processed TGFalpha, it was unable to stimulate receptor activation (FIG. 3E). However, this inactive form of TGFalpha can be converted into the 6 kDa bioactive ligand by incubation with recombinant TACE (FIG. 3F), indicating that RHBDL4-released TGFalpha could be active in vivo if further processed by metalloproteases. Combining the above results, we show that RHBDL4 defines an alternative route for TGFalpha release from cells. The established pathway involves regulated trafficking of pro-TGFalpha by PDZ domain proteins to the plasma membrane, where it is released and activated by TACE. Our data shows that RHBDL4 provides a TGFalpha shedding pathway independent of this trafficking control. This form of TGFalpha moves through the secretory pathway in a soluble but inactive form but can be subsequently activated by metalloproteases. This complex regulation of growth factor trafficking and activation may allow precise spatial and temporal control of EGFR signaling.

### Transactivation

EGFR stimulation in vivo can occur by ‘transactivation’, where GPCR signaling leads to the secondary release of EGFR ligands, which in turn activate the EGFR. The intracellular pathways that lead to EGFR ligand release are actively studied. Indeed, longterm angiotensin treatment (which activates a GPCR) leads to generation of a 37 kDa form of TGFalpha in vivo. Since this form appeared identical to RHBDL4-processed TGFalpha, we investigated whether RHBDL4 might be involved in transactivation.

The peptide hormone bombesin activates the gastrin-releasing peptide receptor, a GPCR expressed in COS-7 cells. Treatment of these cells with bombesin enhanced the BB94-insensitive release of the 37 kDa form of TGFalpha. This response was further enhanced by overexpressing the receptor, confirming that TGFalpha release in response to bombesin was caused by GPCR activation (FIG. 4A). Similar BB94-insensitive activity was induced by PMA (FIG. 4B). All these forms released by BB94-insensitive endogenous activity were indistinguishable from the 37 kDa form generated by RHBDL4 overexpression (FIG. 4B). Although previous studies of transactivation have shown it to be BB94-sensitive, these have primarily assayed the activation of the EGFR. In the light of our data, we suspected that, upon transactivation, RHBDL4 releases an intermediate form of TGFalpha that requires subsequent metalloprotease activation to form the bioactive ligand. Indeed we see direct evidence for this: when ADAMs were not inhibited by BB94, the 37 kDa form of TGFalpha disappeared, in concert with an increase in an 18 kDa form, and the appearance of the 6 kDa bioactive ligand (FIG. 4A). The in vitro cleavage by TACE described above (FIG. 3F) demonstrates that this metalloprotease-dependent trimming of RHBDL4 generated TGFalpha can be catalyzed by TACE.

A central prediction of our model is that the observed BB94-insensitive TGFalpha release would be inhibited by the serine protease inhibitor DCI, a rhomboid inhibitor. This experiment is difficult because robust RHBDL4-triggered release of TGFalpha is detectable only several hours after stimulation (FIG. 4C), but DCI is toxic to cells over a similar time course. To help the cells survive, we expressed the antiapoptotic protein Bch XL. DCI had a strong and specific inhibitory effect on the release of the 37 kDa form of TGFalpha in response to PMA (FIG. 4C). We also tested whether the generation and release of the higher molecular weight form of TGFalpha was inhibited by TAPI-2 (20 μM), BB3103 (20 μM), beta-secretase inhibitor IV (10 μM) and furin inhibitor I (100 μM), but none of these inhibitors of known proprotein convertases had an effect. Overall, these experiments strongly support that EGFR transactivation is triggered by RHBDL4-catalysed shedding of pro-TGFalpha.

### Intramembrane Proteolysis

It has been suggested that the ER is free of most proteases so that newly synthesized proteins that are not yet fully folded are not subject to inappropriate proteolysis. The discovery of RHBDL4 as an ER protease may therefore have significance beyond its role in TGFalpha processing. To our knowledge, signal peptidase and the intramembrane protease SPP, both involved in the processing of ER-targeting signal peptides, are the only previously reported endoproteases in the ER. RHBDL4, which cleaves type I membrane proteins, has complementary activity to SPP, which is specific for type II-orientated TMDs. Therefore both possible orientations of TMDs can be cleaved within the ER. The two enzymes show selectivity for substrate TMDs but they have different modes of regulation: SPP substrates require precleavage by signal peptidase, while RHBDL4 can be activated by GPCR and PKC activity.

### Summary

We teach an alternative pathway for the release of the EGFR activating ligand TGFalpha. The evidence for an essential role of metalloproteases like TACE is overwhelming, and our data do not contradict this since, even after RHBDL4 triggered secretion, soluble TGFalpha is inactive until further modified by TACE (or a related enzyme). Instead our data suggest that GPCR coupled transactivation of the EGFR, increasingly recognized causing pathogenic signaling, is a consequence of rhomboid processing. More broadly, a key principle of EGFR regulation discovered in Drosophila and C. elegans, now appears to be widely conserved, even though mammals have evolved more complex control mechanisms requiring metalloproteases in addition to rhomboids.

### Materials and Methods for Example 1

cDNA constructs. Proteins were all cloned into pcDNA3.1 (Invitrogen). Constructs for mouse RHBDL1, RHBDL2 and RHBDL3 tagged with an N-terminal HA₃-tag had been described previously (14). Similarly, mouse RHBDL4 (IMAGE cDNA clone 3494511) was cloned with an N-terminal HA₃-tag. Note that RHBDL4 (Swiss-Prot accession Q8BHC7) has not been studied so far and has been named previously as rhomboid domain-containing protein 1 (Rhbdd1) by automated annotation. Rhomboid mutants were generated by Quick-Change site-directed mutagenesis (Stratagene) replacing the catalytic serine by alanine. Human pro-TGFalpha (7) was used either untagged or tagged in the pro-peptide (between residue 31 and 32; by a FLAG₃-tag or a FLAG₆-tag). The open reading frame coding mouse TGN46 (IMAGE cDNA clone 3157708), human calnexin (IMAGE cDNA clone 3546389), mouse S1P without pro-peptide (IMAGE cDNA clone 5310414) and mouse TACE without pro-peptide (IMAGE cDNA clone 5705503) were amplified by PCR and cloned downstream of the signal peptide of Drosophila Spitz followed by a linker sequence and the FLAG₃-tag. Mouse gastrin-releasing peptide receptor (IMAGE cDNA clone 40047100) was cloned untagged. The construct TGFalpha-TMD-CNX and TGFalpha-TMD-L₂₃ were generated by overlap extension PCR (30), replacing amino acid 99 to 121 of TGFalpha by residue 482 to 504 from calnexin and 23 leucines respectively. The juxtamembrane poly-His-tag in TGFalpha-H₈ was introduced at position 94 of TGFalpha The construct coding human Bcl-XL was a gift from Seamus Martin and had been described previously (31).

Cell culture and cell-based rhomboid cleavage assay. Cells were propagated in DMEM supplemented with 10% fetal calf serum. COS-7 cells were transfected in 35 mm wells with FuGENE 6 (Roche) as described (7). In brief, 250 ng plasmid encoding the substrate (as indicated in the description of the figures), 25 ng for the rhomboid tested, 50 ng for the GRP receptor and empty plasmid to bring the total DNA to 1 μg was used. For protease titration, 2.5 ng to 250 ng plasmid coding RHBDL4 was used. HeLa and HEK293T were transfected with polyethyleneimine (linear, MW 25000; Polysciences) as described (32) using twice the amount of DNA as used with FuGENE. Transfection efficiency was monitored by co-transfection of pEGFP (Invitrogen). Sixteen hours post transfection, medium was replaced with serum-free medium containing 10 μM BB94 (British Biotech) unless otherwise stated. For activation of endogenous rhomboid activity, phorbol 12-myristate 13-acetate (PMA) (1 μM, from Sigma) or bombesin (100 nM, from Sigma) was added to the cell medium. For inhibitor studies, the indicated protease inhibitors (from Calbiochem), diluted from a stock solution in DMSO, were compared with a carrier only. Medium was harvested typically after 24 to 30 hours; for inhibitor studies using 3,4-Dichloroisocoumarin (DCI) a time course with 0 minutes, 30 minutes and 4 hours was performed (FIG. 4C). Cells were solubilized in SDS-sample buffer and analyzed by SDS-PAGE. EndoH (New England Biolabs) and PNGaseF (New England Biolabs) treatment of SDS-solubilized cell extracts was performed according to the manufacturers instructions. Conditioned media were centrifuged for 10 minutes at full speed in a microfuge to remove cell debris, and subsequently proteins in the supernatant were precipitated by adding trichloroacetic acid (TCA) to 10%. The precipitate was recovered by centrifugation, washed with acetone and dissolved in SDS-PAGE sample buffer and analyzed by SDS-PAGE. Alternatively to TCA precipitation, TGFalpha-H₈ in conditioned medium was captured by metal-chelate chromatography using Ni-NTA agarose beads (Qiagen) in the presence of 20 mM imidazole at pH 8.0. Subsequently beads were washed with 20 mM Tris-Cl pH 8.0, 50 mM imidazole, eluted with SDS-sample buffer and analyzed by SDS-PAGE and Western blotting (see below). Typically, from a 35 mm tissue culture dish, 10% of cell extracts and 20% of tissue culture supernatant were loaded. To increase the sensitivity, for experiments shown in FIGS. 1C, 3E, 4A and 4C, five times the amount of the media fractions were loaded.

Antibodies and siRNA treatment. A polyclonal antibody specific for RHBDL4 was raised by immunizing a rabbit with recombinant GST fusion protein comprising amino acid 238 to 315 of mouse RHBDL4, which was purified on glutathione-sepharose and released by thrombin cleavage of the GST tag. For affinity purification the GST fusion protein was coupled to HiTrap NHS-activated HP (Amersham Biosciences) and used to purify the antibody according to standard protocols. In order to prove antibody specificity, cells were transfected with siRNA (100 nM) using DharmaFECT 1 and 2 (Dharmacon) according to the manufacturers description and analyzed by Western blotting after 4 days incubation. The following target sequences were used 5′-GGACGGCAAUACUACUUUA (R4-01, for HeLa, HEK293T and COS-7), 5′-AGCUCGAGAGAGCAUUACA (hR4-02, for HeLa and HEK293T) and 5′-ACAGCUUGAGAGAGCUUUA (cR4-02, for COS-7). The human and green monkey specific siRNAs were used as controls (hR4-02 for COS-7 and cR4-02 for human cells).

Immunofluorescence. Cells were fixed in methanol at −20° C. for 5 minutes followed by acetone at −20° C. for 45 seconds. Following washing with PBS and blocking with 20% fetal calf serum in PBS, cells were probed with affinity-purified anti RHBDL4 antibody (1:500; see above) and anti BAP31 antibody A1/182 (1:1000; Alexis). After staining with fluorescently labeled secondary antibody (Santa Cruz Biotechnology), slides were analyzed using a Zeiss LSM confocal microscope. Note that fixation conditions were critical and standard PFA fixation and solubilization with Triton X-100 resulted in fragmented inhomogeneous structures.

In vitro TACE assay. Proteins in conditioned medium of a cellular cleavage assay with untagged pro-TGFalpha were desalted by a PD-10 column (Amersham Biosciences) equilibrated with 10 mM Tris-Cl pH 7.4. Samples were incubated with 0.5 μg recombinant mouse TACE (R&D Systems) at 37° C. for 24 hours; after TCA precipitation, pellets were washed in acetone, dissolved in SDS-PAGE sample buffer, and analyzed by Western blotting. Note that the cleavage reaction was very inefficient due to inactivation of recombinant TACE by trace amounts of salt.

EGFR activation assay. Subconfluent A431 cells were grown in serum free medium for 24 hours, followed by incubation with conditioned medium that had been harvested from a cellular RHBDL4-cleavage assay using untagged pro-TGFalpha (see above). After 10 minutes incubation at 37° C., cells were lyzed in SDS-sample buffer and analyzed by Western blotting.

Western blotting. Proteins were analyzed by 4-20% Tris-Glycine gradient gels (Invitrogen) followed by Western blot analysis using either anti FLAG M2-HRP (1:1000; Sigma), anti HA antibody 16B12 (1:1000; Covance), anti actin antibody ab8227 (1:5000; Abcam) or affinity purified polyclonal rabbit antibody anti RHBDL4 (1:4000; see above). In order to detect the 6 kDa form of TGFalpha, proteins were transferred on PVDF membrane with 0.2 μm pore size (Millipore) and probed with 1:100 anti TGFalpha antibody 134A-2B3 (Oncogene). For the detection of phosphorylated EGFR, PVDF membranes were blocked in 3% BSA in TBS-Tween supplemented with 200 μM NaVO₃. Protein was detected with anti phospho-EGFR antibody 9H2 (1:2000, Upstate). Subsequently membranes were stripped and reprobed with the antibody EGFR 1005 (1:1000, Santa Cruz Biotechnology). Bound antibodies were detected by incubation with secondary antibody (Santa Cruz Biotechnology) followed by enhanced chemiluminescence (Amersham Biosciences).

### REFERENCES TO EXAMPLE 1

1. A. Gschwind, O. M. Fischer, A. Ullrich, Nat Rev Cancer 4, 361 (2004).
2. B. Linggi, G. Carpenter, Trends Cell Biol 16, 649 (2006).
3. J. J. Peschon et al., Science 282, 1281 (1998).
4. C. P. Blobel, Nat Rev Mol Cell Biol 6, 32 (2005).
5. R. C. Harris, E. Chung, R. J. Coffey, Exp Cell Res 284, 2 (2003).
6. A. Dutt, S. Canevascini, E. Froehli-Hoier, A. Hajnal, PLoS Biol 2, e334 (2004).
7. J. R. Lee, S. Urban, C. F. Garvey, M. Freeman, Cell 107, 161 (2001).
8. S. Urban, J. R. Lee, M. Freeman, Cell 107, 173 (2001).
9. A. Merlos-Suarez, S. Ruiz-Paz, J. Baselga, J. Arribas, J Biol Chem 276, 48510 (2001).
10. A. Pandiella, M. W. Bosenberg, E. J. Huang, P. Besmer, J. Massagué, J Biol Chem 267, 24028 (1992).
11. J. Arribas et al., J Biol Chem 271, 11376 (1996).
12. J. C. Pascall, K. D. Brown, Biochem Biophys Res Commun 317, 244 (2004).
13. S. Urban, M. Freeman, Mol Cell 11, 1425 (2003).
14. O. Lohi, S. Urban, M. Freeman, Curr Biol 14, 236 (2004).
15. M. K. Lemberg, M. Freeman, submitted (2007).
16. A. Lautrette et al., Nat Med 11, 867 (2005).
17. S. W. Sunnarborg et al., J Biol Chem 277, 12838 (2002).
18. S. C. Li, C. M. Deber, Nat Struct Biol 1, 558 (1994).
19. M. Margeta-Mitrovic, Y. N. Jan, L. Y. Jan, Neuron 27, 97 (2000).
20. K. Horiuchi et al., Mol Biol Cell 18, 176 (2007).
21. B. Meusser, C. Hirsch, E. Jarosch, T. Sommer, Nat Cell Biol 7, 766 (2005).
22. O. M. Fischer, S. Hart, A. Gschwind, A. Ullrich, Biochem Soc Trans 31, 1203 (2003).
23. D. C. Heimbrook, J. W. Wallen, N. L. Balishin, A. Friedman, A. Oliff, J Natl Cancer Inst 82, 402 (1990).
24. N. Prenzel et al., Nature 402, 884 (1999).
25. D. Zhao et al., J Biol Chem 279, 43547 (2004).
26. D. C. Huang, S. Cory, A. Strasser, Oncogene 14, 405 (1997).
27. E. A. Evans, R. Gilmore, G. Blobel, Proc Natl Acad Sci USA 83, 581 (1986).
28. A. Weihofen, K. Binns, M. K. Lemberg, K. Ashman, B. Martoglio, Science 296, 2215 (2002).
29. M. K. Lemberg, B. Martoglio, Mol Cell 10, 735 (2002).
30. S, N. Ho, H. D. Hunt, R. M. Horton, J. K. Pullen, L. R. Pease, Gene 77, 51 (1989).
31. P. Delivani, C. Adrain, R. C. Taylor, P. J. Duriez, S. J. Martin, Mol Cell 21, 761 (2006).
32. Y. Durocher, S. Perret, A. Kamen, Nucleic Acids Res 30, E9 (2002).

### Example 2

### Rhomboid Analysis and Identification of Proteases

In part, embodiments of the invention are based on functional and evolutionary implications of enhanced genomic analysis of rhomboid intramembrane proteases described herein.

### Rhomboid Family Overview

Rhomboids are a recently discovered family of widely distributed intramembrane serine proteases that have diverse biological functions including the regulation of growth factor signalling, mitochondrial fusion, and parasite invasion. Despite their existence in all branches of life, the sequence identity between rhomboids is low, making comprehensive genomic analysis challenging. By combining functional data with sequence alignment we have overcome the difficulties of genomic analysis of such a widespread and diverse enzyme family. We show that robust membrane topology models are very important to detect rhomboids unambiguously, and thereby define rules for rhomboid identification, revising estimates of numbers of proteolytically active rhomboids. We thus identify true active rhomboids, and a number of other inactive proteases. The active proteases are themselves subdivided into secretase and PARL-type (mitochondrial) subfamilies; these have distinct transmembrane topologies. This functionally enhanced genomic analysis leads to novel mechanistic conclusions. Most significantly, it suggests that a given rhomboid can only cleave a single orientation of substrate, and that both products of rhomboid catalysed intramembrane cleavage can be released from the membrane. This genomic analysis provides the first strict definition of rhomboid proteases providing a functionality-based classification. Rhomboids appear more ancient than previously recognised and, contrary to a previous proposal, a rhomboid-type intramembrane protease gene was probably present in the last universal common ancestor of current species.

### Intramembrane Proteolysis

Intramembrane proteolysis has over the last few years become recognised as an important cellular regulatory mechanism. Intramembrane proteases fall into three mechanistic classes, the S2P metalloproteases, the GxGD-type aspartyl proteases, including presenilin/gamma-secretase and SPP, and the rhomboid serine proteases). The rhomboid gene was first discovered in Drosophila, where it was named after an embryonic mutant phenotype. More recently, Drosophila Rhomboid-1 was shown to be the founding member of a class of polytopic membrane proteins conserved throughout evolution. Genetic and cell biological analysis revealed that rhomboids are intramembrane serine proteases. Drosophila Rhomboid-1 cleaves membrane-tethered growth factor precursors, releasing the active form and triggering their secretion; thereby, it is the primary activator of epidermal growth factor receptor (EGFR) signalling. The C. elegans rhomboid ROM1 has similarly been implicated in EGFR control.

In other eukaryotic species much less is known about the role of intramembrane proteolysis by rhomboids but there is evidence for significant functions in a variety of contexts. For example, in the apicomplexan parasites P. falciparum and T. gondii, rhomboids are involved in the shedding of adhesion molecules, and have been implicated in host cell invasion. In the yeast S. cerevisiae, Drosophila and mammals, a subclass of rhomboids located in the inner mitochondrial membrane has recently been the focus of attention. In S. cerevisiae the mitochondrial rhomboid Pcp1 (or Rbd1) controls mitochondrial membrane fusion by cleaving the dynamin-like GTPase Mgm1. Pcp1/Rbd1 is conserved across eukaryotes, and related but not identical functions have been shown for the orthologues in Drosophila (Rhomboid-7) and mice (PARL). Finally, two putative substrates (thrombomodulin and ephrin-B3) for mammalian non-mitochondrial rhomboids were identified by candidate testing, although their physiological significance remains unclear. Thus, numerous industrial applications of embodiments of the invention are described in addition to those which are apparent from the specification.

There has been much recent progress in the molecular understanding of rhomboid function, and how these enzymes perform the unusual cleavage of peptide bonds in the hydrophobic plane of the cellular membrane. Rhomboid activity has been reconstituted in vitro, enabling mechanistic questions to be addressed {Lemberg et al., 2005, EMBO J, 24, 464-72; Maegawa et al., 2005, Biochemistry, 44, 13543-52; Urban and Wolfe, 2005, Proc Natl Acad Sci USA, 102, 1883-8}.

Complementary to this functional analysis, high-resolution structures of the E. coli rhomboid GlpG have recently provided insight into its architecture (Wang et al., 2006, Nature, 444, 179-80; Wu et al., 2006, Nat Struct Mol Biol, 13, 1084-1091). Predictions about how one class of rhomboids act, revealing a dyad between a conserved serine and histidine in their catalytic centre, with subsidiary functions in other domains can be made interview of these studies {Lemberg et al., 2005, EMBO J, 24, 464-72; Wang et al., 2006, Nature, 444, 179-80}. The molecular structure function predictions made in the prior art are, however, hampered by the diversity of the rhomboid family. Many genes have been annotated in the art as rhomboids by BLAST searching, but many false positives are also found, preventing rigorous classification or genomic analysis of this important enzyme family. Although it has been stated that the rhomboids are uniquely conserved among polytopic membrane proteins, sequence similarity over the entire length of distant homologues is actually quite low. In arriving at the invention we have exploited recent understanding of rhomboid structure and mechanism to enhance BLAST-based predictions. From this we derive a new stringent and function-based definition of rhomboids, enabling comprehensive and accurate annotation of genomes. As well as providing the first robust classification of rhomboid proteases, we report conserved inactive rhomboid-like proteins. This functionally enhanced genomic analysis also leads to mechanistic and evolutionary conclusions about rhomboid enzymes. Notably, we disclose that rhomboids can cleave substrates in a single membrane orientation specific manner. We further disclose that rhomboid action can release both N- and C-terminal protein domains from substrates.

### The Minimum Consensus Sequence for Rhomboid Proteases

Rhomboids are widely conserved, but the degree of similarity within the family is quite low; in some cases less then 18%. Despite this crude BLAST searching has been used in the art to identify apparently comprehensive lists of rhomboids in sequenced genomes. We aligned the sequences of all rhomboids studied in mutagenesis experiments to determine the minimum sequence requirements. Alignment of the full-length proteins is unsatisfactory due to the heterogeneity of tails and sequence insertions. Multiple sequence alignment of just the conserved membrane-integral portion shows that although all transmembrane domains (TMDs) can be aligned, substantial conservation is only observed in a few regions, comprising the active site formed by the serine protease motif (GxSx in TMD4 and H in TMD6) and a domain (in the L1 loop and TMD2) with a prominent tryptophan-arginine motif (WR). Recent crystal structures of the E. coli rhomboid GlpG confirm that these residues contribute to the heart of the enzyme. This alignment emphasises that the rhomboid protease consensus is very restricted, making it difficult to predict these proteases by simple primary sequence analysis alone. Notably, similar sequence motifs are found in unrelated polytopic membrane proteins. For instance, a GxS-sequence similar to the rhomboid active site consensus is common in TMD5 of the Sec61/SecY superfamily although it is unlikely to have a prominent functional implication. We conclude that rigorous rhomboid prediction is not possible by simple BLAST searching as has been carried out in the art. We disclose that instead, the overall context of the conserved motifs and the topology of the protein must be taken into account.

### Refining Rhomboid Topology

The need to position conserved rhomboid sequences in the context of overall TMD topology highlights the need to predict rhomboid TMDs with precision. Koonin et al {Koonin et al., 2003, Genome Biol, 4, R19} have proposed that rhomboids adopt three different topologies: bacterial and archaeal rhomboid having a basic six TMD-core; most eukaryotic rhomboids having a seventh TMD fused to the C-terminus (6+1); and a subfamily of eukaryotic rhomboids (named after the human PARL and subsequently shown to be mitochondrial with a seventh TMD fused to the N-terminus (1+6). Confusion arises, however, for the experimentally well-studied PARL homologue in yeast, Pcp1/Rbd1, and the predicted T. gondii orthologue, ROM6, in which six TMDs have been proposed. This would suggest that topology has not been conserved within the PARL subfamily, in turn suggesting that specific topology may not be fundamental to rhomboid function. We therefore decided to re-examine the topology of PARL and its orthologues from mouse, zebrafish, D. melanogaster, C. elegans, T. gondii and S. cerevisiae.

TMD prediction, particularly in polytopic membrane proteins, is imprecise so we compared the results of four TMD-prediction programs (see Materials and methods) {Nilsson et al., 2000, FEBS Lett S, 486, 267-9}.

### TMD Prediction and Comparative Topology Analysis

Rhomboid topology models were constructed by superimposing TMD predictions from four different prediction algorithms on a ClustalW multiple-sequence alignment of homologues and orthologues {Thompson et al., 1994, Nucl. Acids Res., 22, 4673-4680} (using MacVector™7.2.2). Where possible, precise TMD boundaries were based on a comparison with structural information taken from the E. coli rhomboid GlpG {Wang et al., 2006, Nature, 444, 179-80}. As prediction algorithms we used TMHMM version 2.0 (http://www.cbs.dtu.dk/services/TMHMM-2.0/) {Sonnhammer et al., 1998, Proc Int Conf Intell Syst Mol Biol S, 6, 175-82}, HMMTOP version 2.0 (http://www.enzim.hu/hmmtop/index.html) {Tusnady and Simon, 2001, Bioinformatics S, 17, 849-50}, PSORT II (psort.nibb.ac.jp/form2.html) {Gardy et al., 2005, Bioinformatics S, 21, 617-23}, and TMpred (http://www.ch.embnet.org/software/TMPRED_form.html). Although these prediction schemes were initially designed for proteins in the secretory pathway and the mechanism of import of mitochondrial membrane proteins is less well understood, it is expected that translocation mediated recognition of TMDs is based on similar principles making this a robust approach. Not all the algorithms predict all TMDs, but combining these results and superimposing their six TMD-core on the known structure of GlpG supports a universal seven TMD structure for PARL-type rhomboids. Within this framework TMDs that are not predicted by any program, such as TMD2 of C. elegans PARL (ROM5), can nevertheless be clearly aligned, with an aspartate (D), a charged residue not common in TMDs, explaining the prediction failure. Taken together, this comparative analysis alters the predicted number of TMDs in S. cerevisiae Pcp1/Rbd1 and T. gondii ROM6, which has significant implications for rhomboid function (see below).

### A New Classification of Rhomboid Topologies

Modifying previous rhomboid topology models (ibid). We now suggest four different topological classes for rhomboid-like proteins. The basic class of a six-TMD core is found in E. coli GlpG and some eukaryotic rhomboids such as S. cerevisiae Rbd2 (YPL246C). The next class, with Drosophila Rhomboid-1 as its most studied member, has an extra TMD fused to the C-terminus and a variable N-terminal domain. In contrast with the prior art we note that this topology is not unique to eukaryotes: many bacterial rhomboids are predicted to have a clear 6+1 TMD structure. The third class is characterised by a large globular domain inserted into the L1 loop and variations in the active site (see below). Note that all these three classes can have additional globular domains, fused either to the N- or C-termini. Finally, the PARL-subfamily has an extra TMD fused to the N-terminus of the rhomboid core, thereby changing the position of the catalytic residues to TMD5 and TMD7 (instead of TMD4 and TMD6 in other rhomboids); PARLs also have long N-terminal extensions. Taken together this clearly shows that substantial diversification between different rhomboid proteases has occurred. The invention facilitates study of the family, for example to determine more fully how extra TMDs affect the structure and function of more complex rhomboids.

### Method of Identifying Rhomboid Proteases

In order to generate a complete list of true rhomboid proteases for significant organisms and, equally importantly, to remove falsely annotated genes, we have exploited our new definitions of rhomboids. We propose defining as rhomboids only proteins that are predicted to be catalytically active (see below). The steps in this process were as follows: 1) homology search with PSI-BLAST, using the core domain of unambiguous rhomboid proteases; 2) construction of a topology model; 3) examine if the minimal rhomboid-protease consensus (GxSx & H) fits the 6-TMD protease core (i.e. do the catalytic residues lie in a topologically appropriate position?); and 4) look for the presence of additional conserved features, such as the residues characteristic of L1/TMD2. In order not to lose any more distant related but bona fide rhomboids, the last step (step 4) may optionally be omitted. A complete list of the rhomboid proteases thus defined in humans, mouse, zebrafish, Drosophila, C. elegans, S. cerevisiae, P. falciparum, T. gondii, Arabidopsis, and rice (O. sativa) is given in. Revising previous suggestions, we find five putative rhomboid proteases in humans, mice and zebrafish (D. rerio), six in Drosophila, six in P. falciparum, two in C. elegans, 13 in Arabidopsis and 12 in rice (O. sativa). In agreement with previous reports, we find six rhomboid homologues in T. gondii and two in S. cerevisiae. This stringent approach has allowed us to remove a significant number of apparently unrelated genes (two each in human and mouse, and six in Arabidopsis; see Table A for details); and a number of related inactive homologues that lack key catalytic residues (see below). Importantly, we are confident that all rhomboid proteases in these species have been identified according to the present invention.

### Rhomboid Nomenclature

In conjunction with this genome-wide analysis, we propose some rationalisation of rhomboid nomenclature to avoid future confusion. We propose keeping established names of genes that have been significantly studied, with the exception that running numbers in the name should be based on their appearance in the literature, which leads to a few alterations. Based on functional differences, we further suggest distinguishing PARL-type and secretase-type rhomboids. Since all species analysed so far have only one copy of the PARL subfamily, the scope for confusion is not great, so we suggest that previously used names such as Drosophila Rhomboid-7 and S. cerevisiae Pcp1 be retained, as long as reference is made to these being of the PARL subfamily.

### Phylogenetic Relationship of Eukaryotic Rhomboid Homologues

Having established a complete list of rhomboid proteases and putative inactive homologues for various eukaryotes, we next questioned their phylogenetic relationships. We were prompted to revisit this by the observation that the two rhomboids in S. cerevisiae, Pcp1/Rbd1 and Rbd2, localize to mitochondria and Golgi apparatus respectively, yet had both been placed in the PARL subfamily, which is now known to be mitochondrial. We wondered whether by using stringent alignments of functionally important regions of rhomboids, we could develop a phylogenetic tree that reflected the current understanding of rhomboids more fully, including the known subcellular localization.

### Multiple-Sequence Alignment and Phylogenetic Analysis

We obtained 82 sequences for rhomboid proteases and rhomboid-like proteins. Based on our topology model, we artificially spliced together the conserved regions (C-terminal 13 amino acids of L1, TMD2, TMD4 and TMD6 for secretase-type rhomboids; C-terminal 13 amino acids of L2, TMD3, TMD5 and TMD7 for PARL-type rhomboids). In total 86 amino acids were aligned and a phylogeny tree was constructed based on the UPGMA analysis using MacVector™7.2.2 software. To test the support of individual clades 1000 bootstrap replicas were performed.

### Prediction of Sub-Cellular Localization and Protein Search for Conserved Protein Domains

Sequences were analyzed by TargetP 1.1 (http://www.cbs.dtu.dk/services/TargetP/) {Emanuelsson et al., 2000, J Mol Biol S, 300, 1005-16}, ChloroP (http://www.cbs.dtu.dk/services/ChloroP/) {Emanuelsson et al., 1999, Protein Sci S, 8, 978-84}, MITOPRED (http://bioinformatics.albany.edu˜mitopred/) {Guda et al., 2004, Bioinformatics S, 20, 1785-94}, PSORT II (http://psort.nibb.ac.jp/form2.html) {Gardy et al., 2005, Bioinformatics S, 21, 617-23} and rps-BLAST (http://www.ncbi.nlm.nih.gov/BLAST/) {Marchler-Bauer et al., 2002, Nucleic Acids Res S, 30, 281-3}. Bootstrap analysis of our consensus tree shows that indeed all PARL-type rhomboids fall into one clade, but now places the second yeast rhomboid Rbd2 in a different clade. This analysis also separated the non-PARL rhomboids into many subgroups, indicating a substantial diversification. To enable a better comparison between more closely related species, we analyzed separately parasites and plants, which have more divergent rhomboids. This simplified phylogenetic tree shows four major clades: the PARL-type rhomboids; a major clade consisting of bona fide rhomboids (secretase-type A); a second clade of secretase rhomboids (B-type); and finally, a clade of more distantly related rhomboids that lack catalytic residues.

A few rhomboid homologues did not fit into any of these groups: by virtue of having mutated core residues, they are predicted to be catalytically inactive but they do not cluster with the other inactive species. These include, for example C. elegans C48B4.2 (formerly ROM2 by automated annotation), and At5g38510 and KOMPEITO from Arabidopsis. These do not form a coherent phylogenetic group and we believe them to be relatively recent mutations of active rhomboids; we refer to them simply as inactive rhomboid homologues but do not further classify them. We now outline some features of the rhomboid-like groups and subfamilies and discuss the implications of this tree.

### PARL-Type Rhomboids

Members of this subfamily all have the 1+6 TMD topology discussed above. The branching within the PARL subfamily reflects the species tree indicating that our analysis is correct and reflects the phylogenetic relation of rhomboids. The biological significance of this subfamily is supported by their high degree of overall homology, their identical topology, and their predicted mitochondrial localisation. We therefore suggest that PARL-type rhomboids may have derived from a common ancestor. The substrate of PARL-type rhomboids in S. cerevisiae, Drosophila and mouse appears to have been conserved suggesting that their function is related.

### Secretase-Type Rhomboids

The secretase subfamily is so called because all its studied members are located in the secretory pathway; it contains the majority of eukaryotic rhomboids. Although the homology within this subfamily is quite high, significant differences exist and we find these proteins split into two clades. Secretase-A rhomboids have a 6+1 TMD topology described above, while secretase-B rhomboids have the 6 TMD core only. Note, however, that we find one exception in each class: Drosophila Rhomboid-6 has 6 TMDs, and Arabidopsis RBL12 is predicted to have 6+1. These could represent annotation defects, but they may imply that the TMD topology distinction between the secretase-A and -B rhomboids is not absolute. Another notable distinction between the A and B classes is that the WR-motif in L1 is strictly conserved in the A class, whereas with the exception of the more distant Arabidopsis RBL12, the B-class has only the conserved arginine. There are also clear distinctions in the sequence around the catalytic serine: there is a highly conserved GxSxGVYA sequence in the A-class, compared to a slightly less rigid consensus of GxSxxxF in the B-class. An interesting variation is observed in the first x-position of all vertebrate secretase rhomboids accessible by the ENSEMBL genome browser: with a glycine (G) in RHBDL1 orthologues, an alanine (A) in RHBDL2 orthologues, a serine (S) in RHBDL3 orthologues and a phenylalanine (F) in RHBDL4 orthologues. We teach that this position influences the activity or substrate specificity.

There has been much diversification within the secretase-A class of vertebrate rhomboids but significant relationships can nevertheless be inferred. All Drosophila secretase rhomboids (Rhomboids-1, -2, -3, -4 and -6) fall into the secretase-A class. Consistent with their demonstrated common function in EGFR control, Rhomboids-1, -2 and -3 are the most closely related.

Rhomboid-4 has a role in EGFR control and is more distantly related. Rhomboid-6 is the most distant Drosophila secretase rhomboid and interestingly is the only one with no detectable function in EGFR control.

### Identification of RHBDL4 Like Rhomboids

The secretase-B rhomboids represent a previously unrecognised class. It contains S. cerevisiae Rbd2, and a group of orthologous rhomboids from human, mouse and zebrafish. These orthologues are the founding members of a subclass of rhomboids, which we name after mammalian RHBDL4. RHBDL4-like rhomboids are found in all chordate genomes annotated by ENSEMBL, and in Arabidopsis (Arabidopsis RBL10 is a clear orthologue of vertebrate RHBDL4) and rice. Despite the prediction of mitochondrial targeting (TargetP and MitoPred, see above for details), immunofluorescence analysis in mammalian tissue culture cells reveals that RHBDL4 is localised in the secretory pathway. Based on these results we show that the RHBDL4-like rhomboids are a distinct subclass of rhomboids within the secretase-B class.

The wide distribution of the RHBDL4 group, combined with their presence with yeast Rbd2 within the secretase-B class, the only secretase-type rhomboid in yeast, suggests that this subclass may have evolved early. The observation that its members have only the core 6 TMDs is also consistent with them resembling an ancestral precursor. The more complex eukaryotic rhomboids may have derived from such a simple rhomboid, an ancient form that might have been lost in nematodes and insects. This would make rhomboids a rare case where the topology appears to have evolved by attachment of non-homologous TMDs, instead of by the more typical internal gene duplication or non-covalent oligomerisation.

Many genes have been annotated as rhomboids by BLAST searching (Koonin et al., 2003, Genome Biol, 4, R19) and a hidden Markov model (PF01694), but many false positives are also found (see Table A). The rhomboid protease consensus is very restricted, making it difficult to predict these proteases by simple primary sequence analysis alone. For a rigorous rhomboid prediction functional data and the context of sequence motifs and the topology of the protein must be taken into account. Based on the position of the catalytic residues we define two rhomboid subfamilies:

1.) secretase-type rhomboids with the catalytic GxSx in TMD4 and the histidine in TMD6, which both have an out-to-in orientation;
2.) mitochondrial PARL-type rhomboids with the active site residues in TMD5 and TMD7, which both have an in-to-out orientation.

In order to generate a complete list of rhomboid proteases in the human and mouse secretory pathway and, equally importantly, to remove falsely annotated genes, we have exploited the rhomboid consensus enhanced by mutagenesis studies and our new topology classification. We define secretase rhomboids as only proteins that are predicted to be catalytically active and have the catalytic motif GxSx in TMD4 and the catalytic histidine in TMD6.

The steps in this process were as follows: 1) homology search with PSI-BLAST, using the core domain of unambiguous rhomboid proteases; 2) construction of a topology model; 3) examination whether the minimal rhomboid-protease consensus (GxSx and H) are in TMD4 and TMD6.

Optionally the further step of: 4) look for the presence of additional conserved features, such as the residues characteristic of L1/TMD2 (see text and FIG. 5) is also applied.

Revising previous suggestions, we show five rhomboid proteases in humans and mice: four secretase-type and one PARL. This stringent approach has allowed us to remove two inactive rhomboid homologues that lack key catalytic residues and two completely unrelated genes, which had been previously suggested to be rhomboids (e.g. see Koonin et al. (2003) above) or automated annotation (see Table A for details).

Our analysis clearly shows that these rhomboid-like genes RHBDF1 and RHBDF2 are proteolytically inactive proteins. Moreover, our analysis identifies the distant related RHBDL4 (with less than 18% sequence identity to Drosophila Rhomboid-1) as secretase-type rhomboid. In the previous reports by Koonin et al. (2003), the mouse equivalent had been suggested to be a putative rhomboid related to PARL. The previous identification was only based on BLAST-search, which is not able to discriminate between rhomboid-like proteolytically inactive proteins and such distantly related rhomboid proteases. The previous phylogenetic analysis aiming to support their findings was based on an imprecise sequence alignment that failed to reveal a biologically meaningful classification. Likewise two secretase-type rhomboids mouse RHBDL4 and S. cerevisiae Rbd2 were previously wrongly classified as PARL-type rhomboids, despite their secretase-type topology and their cellular localization to the secretory pathway (e.g. Huh et al., 2003, Nature S, 425, 686-91). We, however, observe that bootstrap analysis of our more restrictive sequence alignment places RHBDL4 as sub-group of the secretase-type rhomboids and not the PARL family.

### RHBDL4 Consensus

Multiple-sequence alignment of the conserved region according to structure-based TMD prediction of active rhomboids from human (Homo sapiens, Hs), mouse (Mus musculus, Mm), zebrafish (Danio rerio, Dr), Drosophila melanogaster (Dm), Drosophila pseudoobscura (Dp), Caenorhabditis elegans (Ce), Saccharomyces cerevisiae (Sc), Toxoplasma gondii (Tg), Plasmodium falciparum (Pf), Arabidopsis thaliana (At) and rice (Oryza sativum, Os). For accession numbers see below. Based on phylogenetic analysis, the sequences are classified into secretase-type (A, B and other) and PARL-type. For secretase rhomboids the C-terminal portion of L1, TMD2, TMD4 and TMD4 were used for the alignment; for PARL and its orthologues the topological equivalent portion of L2, TMD3, TMD5 and TMD7 are shown; the junctions of artificial splices are indicated by triangles. Background colour reflects the degree of identity/similarity of sequence alignment (100%, red; 90-99% light-red, 80-89%, yellow; 50-79%, dark grey; 30-49%, light grey); the key catalytic residues (GxSx and H) are highlighted; TMDs are underlined.

### Accession Numbers:

For human, mouse and Arabidopsis rhomboids see Table A; for details of the rice genes see MIPS plant genome database (http://mips.gsf.de/projects/plants/). The accession numbers for zebrafish (D. rerio, Dr) RHBDL1 is (ENSEMBL:ENSDARP00000082440) Dr RHBDL2 is (Swiss-Prot:Q7ZUN9); Dr RHBDL3 is (Swiss-Prot:Q566N3); Dr RHBDL4 is (Swiss-Prot:Q568J3); Dr PARL is (ENSEMBL:ENSDARP00000011733); D. melanogaster (Dm) Rhomboid-1 is (Swiss-Prot:P20350); Dm Rhomboid-2 is (Swiss-Prot:Q86P37); Dm Rhomboid-3 is (Swiss-Prot:Q9W0F8); Dm Rhomboid-4 is (Swiss-Prot:Q9VYW6); Dm Rhomboid-6 is (Swiss-Prot:Q86BL6); Dm PARL is (Swiss-Prot:Q9V641); D. pseudoobscura (Dp) Rhomboid-1 is (GenBank:EAL31292); Dp Rhomboid-2 is (GenBank:EAL3128); Dp Rhomboid-3 is (GenBank:EAL31296); Dp Rhomboid-4 is (GenBank:EAL32611); Dp Rhomboid-6 is (GenBank:EAL33827); Dp PARL is (GenBank:EAL25960); C. elegans (Ce) ROM1 is (Swiss-Prot:Q19821); Ce PARL (ROM5) is (GenBank:AAF60768); S. cerevisiae (Sc) Rbd2 is (Swiss-Prot:Q12270); Sc PARL (Pcp1/Rbd1) is (Swiss-Prot:P53259); T. gondii (Tg) ROM1 is (Swiss-Prot:Q696L6); Tg ROM2 is (Swiss-Prot:Q695T9); Tg ROM3 is (Swiss-Prot:Q6IUY1); Tg ROM4 is (Swiss-Prot:Q695T8); Tg ROM5 is (Swiss-Prot:Q6GV23); Tg ROM6 is (Swiss-Prot:Q2PP52); P. falciparum (Pf) ROM1 is (GenBank:AAN35734); Pf ROM3 is (GenBank:CAD51095); Pf ROM4 is (GenBank:CAD51434); Pf ROM6 is (GenBank:CAD52576); Pf ROM7 is (GenBank:CAD52703); Pf ROM9 is (GenBank:NP_{—}703495).

TABLE A
Genome-wide analysis of rhomboid homologues in human and mouse.

| | | | Swiss-Prot | | |
|---|---|---|---|---|---|
| Species | Proposed name | Gene ID | accession | Synonyms | Basis for proposed name |
| Human | RHBDL1 | 9028 | O75783 | RHBDL, | published by {Urban et al., 2001, Cell, 107, 173-82}; |
| | | | | veinlet-like 1, RRP1 | alternative RHBDL, published by {Pascall and Brown, 1998, FEBS |
| | | | | | Letters, 429, 337-340} |
| | RHBDL2 | 54933 | Q9NX52 | veinlet-like 2, | published {Urban et al., 2001, Cell, 107, 173-82} |
| | | | | RRP2 | |
| | RHBDL3 | 162494 | Q495Y4 | ventrhoid, RHBDL4, | mouse orthologue published by {Lohi et al., 2004, Curr Biol, 14, 236-41}; |
| | | | | veinlet-like 3, | alternative ventrhoid, published by {Jaszai and Brand, 2002, Mech |
| | | | | RRP3, RHBDL3 | Dev, 113, 73-7}; |
| | | | | | RHBDL3 preferred for consistency |
| | RHBDL4 | 84236 | Q8TEB9 | Rhbdd1 | in this study; alternative Rhbdd1 by automated annotation |
| | PARL | 55486 | Q9H300 | PSARL | published by {Pellegrini et al., 2001, J Alzheimers Dis, 3, 181-190} |
| | — | 64285 | Q4TT59 | RHBDF1 | automated annotation; not predicted to be a rhomboid protease |
| | | | | | consensus mismatch: |
| | | | | | GPAG in TMD4 |
| | — | 79651 | | RHBDF2, | automated annotation and {Koonin et al., 2003, Genome Biol, 4, |
| | | | | veinlet-like 6 | R19}; not predicted to be a rhomboid protease |
| | | | | | consensus mismatch: |
| | | | | | GPAG in TMD4 |
| | — | 57414 | | Rhbdd2 | automated annotation; not predicted to be a rhomboid protease |
| | | | | | consensus mismatch: |
| | | | | | no TMD2-signature; GFTP instead of GxSx in putative TMD4; |
| | | | | | N instead of H in putative TMD6 |
| | — | 25807 | | Rhbdd3 | automated annotation; not predicted to be a rhomboid protease |
| | | | | | consensus mismatch: |
| | | | | | no TMD2 signature; GLSS in putative TMD4; no H in putative TMD6 |
| Mouse | RHBDL1 | 214951 | Q8VC82 | veinlet-like 1 | published by {Lohi et al., 2004, Curr Biol, 14, 236-41} |
| | RHBDL2 | 654339 | | veinlet-like 2 | published by {Urban and Freeman, 2003, Mol Cell, 11, 1425-34} |
| | RHBDL3 | 246104 | P58873 | veinlet-like 3 | published by {Lohi et al., 2004, Curr Biol, 14, 236-41}; |
| | | | | | alternative ventrhoid, published {Jaszai and Brand, 2002, Mech Dev, |
| | | | | | 113, 73-7}. |
| | | | | | RHBDL3 preferred for consistency |
| | RHBDL4 | 76867 | Q8BHC7 | Rhbdd1 | in this study; alternative Rhbdd1 by automated annotation and |
| | | | | | wrongly annotated as PARL-type rhomboid by {Koonin et al., 2003, |
| | | | | | Genome Biol, 4, R19}; |
| | PARL | 381038 | Q5XJY4 | PSARL | published {Cipolat et al., 2006, Cell, 126, 163-75}; orthologue to |
| | | | | | human PARL |
| | — | 13650 | Q6PIX5 | RHBDF1 | automated annotation; not predicted to be a rhomboid protease |
| | | | | | consensus mismatch: |
| | | | | | GPAG in TMD4 |
| | — | 217344 | Q80WQ6 | RHBDF2, | automated annotation; not predicted to be a rhomboid protease |
| | | | | rhomboid-like | consensus mismatch: |
| | | | | protein 6 | GPAG in TMD4 |
| | — | 215160 | | Rhbdd2 | automated annotation; not predicted to be a rhomboid protease |
| | | | | | consensus mismatch: |
| | | | | | no TMD2-signature; GFTP instead of GxSx in putative TMD4; |
| | | | | | N instead of H in putative TMD6 |
| | — | 279766 | | Rhbdd3 | automated annotation; not predicted to be a rhomboid protease |
| | | | | | consensus mismatch: |
| | | | | | no TMD2 signature; GLSG in putative TMD4; no H in putative TMD6 |

### Functional Implications of the New Rhomboid Classification

The identification of an extra TMD in all members of the PARL subfamily has caused us to re-evaluate aspects of the published experimental literature and turns out to have profound mechanistic consequences for proteolysis by all rhomboids. The additional TMD shifts the serine protease active site residues from TMD4 and TMD6 in other rhomboids to TMD5 and TMD7. In conjunction with the topology of mitochondrial import implied by the cleaved mitochondrial import signal sequence, the 1+6 TMD structure suggests that the PARL active site has the opposite orientation within the membrane to other rhomboids.

The catalytic GxSx and histidine of secretase rhomboids are located in TMDs 4 and 6 which both are of out-to-in orientation. In contrast, these catalytic motifs in PARLs occur in in-to-out TMDs. Crucially, there is a corresponding inversion of substrate orientation: PARL substrates have an Nᵢₙ/Cₒᵤₜ topology, but secretase rhomboids cleave type I membrane proteins (Nₒᵤₜ/Cᵢₙ). This striking inversion of the active sites of PARLs, and the correlation with inverted substrates has not been apparent until now because of the failure to detect all the TMDs in S. cerevisiae PARL (Pcp1/Rbd1) (see above). This revised topology strongly suggests that all rhomboids can cleave only one substrate orientation.

Examination of the active sites and substrates of PARL and secretase rhomboids also suggests another important mechanistic conclusion. The PARL active sites are predicted to lie close to the matrix side of the membrane (topologically equivalent to the cytoplasm), but the released fragment of the substrate is the intermembrane space (IMS) domain. That is, the cleaved fragment with the long TMD remnant is released. On the other hand, the active site of secretase type rhomboids is close to the other side of the membrane—the luminal or extracellular side, which is topologically equivalent to the IMS; the released fragment of all known substrates of these rhomboids is the side with the short TMD remnant. Therefore both halves of rhomboid cleaved substrates can be released from the membrane. This raises the intriguing possibility that in some cases, rhomboid cleavage may lead to bidirectional signalling, for example simultaneously releasing substrate domains into the cytoplasm and the lumen/extracellular space. This could have profound biological consequences.

### Summary

Recent functional and structural advances in our understanding of rhomboid proteases highlight key domains in the protein sequence. By focusing on these domains, we have remodeled the proposed phylogenetic tree of rhomboid-like genes. In this paper we have focused on the functional and possible evolutionary consequences of this enhanced genomic analysis. Our summary conclusions are as follows.

A. Simple primary sequence comparison (e.g. BLAST or PSI-BLAST) is insufficient to predict rhomboids with high confidence. A topological prediction of the TMD structure is needed as well, which is provided herein.

B. We define four topological classes of rhomboids by virtue of the number and position of TMDs, their orientation in the membrane, and the existence of characteristic extramembrane domains. To our knowledge rhomboids are the first example where topology of a membrane protein has evolved by the covalent fusion of a single TMDs to a conserved core. Although the overall function of this protease core is expected to be conserved, the structural and functional implication of these extra MDs is of interest.

C. We define true rhomboids as being active proteases (and those which are predicted to be active by virtue of their sequence). There are numerous rhomboid-like proteins that are missing catalytically important active site residues. We propose that these not be called rhomboids.

D. Our analysis allows us to predict for the first time the number of rhomboids in sequenced genomes. We therefore revise the number in several species, including humans. This reduces the number of intramembrane proteases for mouse and human to 13 (five rhomboids, one S2P, and seven GxGD-type), instead of 16 as previously suggested.

E. We find four major phylogenetic clades of eukaryotic rhomboid-like proteins: secretase-type, which are divided into A and B classes; PARLs, the mitochondrial subfamily; and finally inactive rhomboid homologues (which we no longer define as true rhomboids). Rhomboids from plants and apicomplexan parasites are too divergent to incorporate fully into these four clades.

F. This genomic analysis suggests significant new areas of study and leads to substantial functional conclusions. Moreover, the topology that we report for all PARL-type rhomboids leads to two major mechanistic conclusions. The first is that a given rhomboid can probably only cleave one orientation of substrate TMD. The second is that both products of a rhomboid-catalysed transmembrane cleavage can leave the membrane, raising the possibility of bidirectional signalling by rhomboids.

G. The revised phylogeny of rhomboids, based on functional and structural data suggests that rhomboids are more ancient that previously thought, with an ancestral rhomboid-like gene being present in the last universal common ancestor of all organisms. Genomic analysis identifies an extant rhomboid, conserved between, yeast, plants and vertebrates, with the most basic 6 TMD domain architecture, which we predict to resemble an ancestral template for all eukaryotic rhomboids. It was previously proposed that rhomboids have spread through evolution by several independent horizontal gene transfer events between species. On the basis of our more rigorous functionally based analysis, we believe that a model of primarily vertical evolution from an ancestral gene is now the more parsimonious conclusion.

### Methods to Example 2

### Sequence Data

Rhomboid sequences were retrieved by BLAST- and PSI-BLAST search {Altschul et al., 1997, Nucleic Acids Res, 25, 3389-402} from the NCBI database (http://www.ncbi.nlm.nih.gov/BLAST/), from the ENSEMBL genome browser (http://www.ensembl.org/index.html) and the MIPS plant genome database (http://mips.gsf.de/projects/plants/).

### WEB SITE REFERENCES

http://www.ncbi.nlm.nih.gov/BLAST/; The National Center for Biotechnology Information
http://www.ensembl.org/index.html; The ENSEMBL Genome Browser
http://mips.gsf.de/projects/plants/; The Munich Information Center for Protein Sequences Plant Genome
http://www.cbs.dtu.dk/services/TMHMM-2.0/; TMHMM Prediction of Transmembrane Helices in Proteins
http://www.enzim.hu/hmmtop/index.html; HMMTOP Prediction of Transmembrane Helices and Topology of Proteins
http://psort.nibb.ac.jp/; database for the prediction of protein localization sites in cells and TMD topology
http://http://www.ch.embnet.org/software/TMPRED_form.html; TMpred Prediction of Transmembrane Regions and Orientation
http://www.cbs.dtu.dk/services/TargetP/; TargetP prediction of subcellular location
http://www.cbs.dtu.dk/services/ChloroP/; ChloroP prediction of chloroplast transit peptides
http://bioinformatics.albany.edu/˜mitopred/; MITOPRED Prediction of Mitochondrial Proteins

### Example 3

### Transactivation Via Alternate Ligands

In the above examples the transactivation/RHBDL4 cleavage is typically mediated by exemplary ligand TGFalpha; in this example alternate ligand is demonstrated as RHBDL4 substrate via the biological demonstration of transactivation. In this example the transactivating ligand/RHBDL4 substrate is HB-EGF.

Treatment with bombesin (bbs) of COS-7 cells overexpressing the bombesin receptor stimulated HB-EGF secretion is shown in FIG. 6. Experiment is performed as in FIG. 4a (except that HB-EGF harbouring an N-terminal FLAG3-tag was used as substrate). In difference to TGFalpha, substantial HB-EGF shedding by ADAM proteases was observed in unstimulated cell (sensitive to BB94, compare lane 1 and 3).

Bombesin treatment enhanced HB-EGF release (bbs, lane 2 and 4). In contrast to prior teachings, this shows insensitivity to BB94 (20 μM). These forms released by BB94-insensitive endogenous activity (i.e. ADAM proteases independent) were indistinguishable from HB-EGF released upon RHBDL4 overexpression, demonstrating RHBDL4 mediation of HB-EGF mediated transactivation.

### Example 4

### In Vitro Assay of RHBDL4

### Protein Expression and Detergent Solubilisation of RHBDL4:

To produce recombinant RHBDL4, a RHBDL4-purification tag fusion protein is expressed and solubilised with detergent appropriate for in vitro activity assay. In this example, C-terminally His6-tagged mouse RHBDL4 is expressed in E. coli BL21-Gold(DE3) cells harbouring the expression vector and the extra plasmid pRARE2 (Novagen) as described for human RHBDL2 (Lemberg, 2005, EMBO vol 24 pp 464-472).

After the protein expression, cells are disrupted and membranes containing the recombinant RHBDL4 are harvested by centrifugation as had been described (Lemberg, 2005 above).

Subsequently the recombinant protein is solubilised with the detergent Triton-X 100 and tested for activity. To this end, the rhomboid substrate is either incubated directly with crude detergent-solubilised membrane fractions containing rhomboids or a pure protease fraction obtained after affinity purification, as has been demonstrated for the bacterial homologues GlpG and YqgP (see Lemberg et al, EMBO Journal 2005 which is expressly incorporated herein by reference. Specifically, the method sections cited in this text are referred to).

### RHBDL4 Protease Assay:

Radiolabelled substrate, such as the substrate TMD, is generated by cell-free in vitro translation using wheat germ extract and [35S]methionine as, had been described (Lemberg and Martoglio, 2003 Anal Biochem. vol 319 pp 327-31).

In this example a substrate corresponding to an N-terminal methionine plus residues 224 to 272 of Drosophila Gurken is used. Other substrate TMDs such as human TGFalpha, human HB-EGF, Drosophila Spitz may be used instead.

For the cleavage assay, 1-4 μl in vitro translation mix or 50-200 μg/ml recombinant substrate are added to a 40 μl-reaction containing recombinant RHBDL4 (e.g. about 1-5 μg) in 50 mM HEPES/NaOH, pH 7.4, 10% glycerol and 50 mM EDTA. Samples are incubated at 30° C. and subsequently the cleavage reaction is analyzed by SDS-PAGE as described (Lemberg, 2005 above).

### Example 5

### RHBDL4 Assay

FIG. 7 shows the results of an in vitro activity assay with recombinant mouse RHBDL4. In vitro translated substrate comprising the transmembrane domain of Drosophila Gurken was incubated with a Triton-X 100 solubilised membrane fraction from E. coli with recombinant mouse RHBDL1 and RHBDL4 and human RHBDL2 as indicated. The substrate was cleaved, as indicated by the decreased amount of intact substrate band. This was inhibited with the serine protease inhibitor dichloroisocoumarin (DCI), known to block the catalytic effect of rhomboids.

This surprisingly shows that RHBDL4 can cleave a generic rhomboid substrate with an apparently similar activity to other rhomboids.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method of identifying a modulator of RHBDL4, said method comprising
(i) providing a first and second sample of cells
(ii) contacting said first sample of cells with a candidate modulator of RHBDL4
(iii) measuring epidermal growth factor receptor (EGFR) transactivation in said first and second samples of cells,
wherein a difference between the transactivation measured in said first and second samples of cells identifies said candidate modulator of RHBDL4 as a modulator of RHBDL4.

2. The method according to claim 1, wherein an increase in transactivation in said first sample of cells relative to said second sample of cells identifies said modulator as a candidate activator of RHBDL4.

3. The method according to claim 1, wherein a decrease in transactivation in said first sample of cells relative to said second sample of cells identifies said modulator as a candidate inhibitor of RHBDL4.

4. The method according to any of claim 1, wherein said transactivation is measured by assessing the level of BB94-insensitive release of EGFR ligand from said cells.

5. The method according to claim 4, wherein said EGFR ligand is the 37 kDa form of TGFalpha.

6. The method according to claim 5 wherein said 37 kDa form of TGFalpha is detected via an amino acid sequence tag.

7. A method of inducing epidermal growth factor receptor (EGFR) transactivation in a system, said method comprising increasing RHBDL4 activity in said system.

8. The method according to claim 7 wherein said RHBDL4 activity induces shedding of pro-TGFalpha.

9. A method of activating RHBDL4 in a system comprising activating protein kinase C (PKC) in said system.

10. Use of a siRNA against RHBDL4 in the manufacture of a medicament for a disease associated with EGFR transactivation.

11. The use according to claim 10, wherein said disease is cancer, kidney disease or cardiovascular disease.

12. The use according to claim 11, wherein said cancer is breast cancer.

13. The use according to claim 10, wherein said siRNA comprises the sequence of at least one of SEQ ID NO:1, SEQ ID NO:2 or SEQ ID NO:3.

14. A method of treating cancer, kidney disease or cardiovascular disease comprising administering to a subject an effective amount of a siRNA wherein said siRNA comprises the sequence of at least one of SEQ ID NO:1, SEQ ID NO:2 or SEQ ID NO:3.

15. The method according to claim 14, wherein said disease is breast cancer.

16. Use of recombinant or purified RHBDL4, or a catalytically active fragment thereof, as a protease.

17. Use of recombinant or purified RHBDL4, or a catalytically active fragment thereof, as a rhomboid secretase protease.

18. Use of recombinant or purified RHBDL4, or a catalytically active fragment thereof, in the cleavage of a polypeptide transmembrane domain.

19. Use of recombinant or purified RHBDL4, or a catalytically active fragment thereof, in the transactivation of EGFR.

20. Use of recombinant or purified RHBDL4, or a catalytically active fragment thereof, in the release of a substrate polypeptide from a membrane.

21. The use according to claim 20 wherein each of the cleavage products of said substrate polypeptide are released from the membrane.

22. A method of releasing a substrate polypeptide from a membrane, said method comprising contacting said substrate polypeptide with recombinant or purified RHBDL4 or a catalytically active fragment thereof.

23. The method according to claim 22, wherein the polypeptide is cleaved by the RHBDL4 and each of the substrate polypeptide cleavage products is released from the membrane.

24. The method according to claim 22, wherein said substrate polypeptide is a TGFalpha polypeptide.

25. A method of processing pro-TGFalpha, said method comprising contacting pro-TGFalpha with recombinant or purified RHBDL4 protein, or a catalytically active fragment thereof.

26. A method of preparing active TGFalpha ligand comprising processing pro-TGFalpha according to claim 25, and further comprising the step of contacting said processed TGFalpha with a metalloprotease.

27. The method according to claim 26, wherein said metalloprotease is an ADAM family metalloprotease.

28. The method according to claim 27, wherein said metalloprotease is TACE.

29. A method of identifying a modulator of RHBDL4 protease, said method comprising
(i) providing a first and second sample of RHBDL4 protease or a catalytically active fragment thereof;
(ii) contacting said first sample of RHBDL4 protease or catalytically active fragment thereof with a candidate modulator of RHBDL4; and
(iii) measuring cleavage of a RHBDL4 substrate by said first and second samples of RHBDL4 protease or catalytically active fragment thereof,
wherein a difference between the cleavage measured in said first and second samples of RHBDL4 protease or catalytically active fragment thereof identifies said candidate modulator of RHBDL4 as a modulator of RHBDL4.

30. The method according to claim 29, wherein said substrate comprises residues 224 to 272 of Drosophila Gurken, and wherein said cleavage is monitored by SDS-PAGE.

31. The method according to claim 29, wherein a decrease in the protease activity determined in the first sample relative to the second sample indicates that said modulator is an inhibitor of RHBDL4 protease.

32. A method of inhibiting transactivation of an ErbB family receptor in a system, said method comprising inhibiting RHBDL4 in said system.

33. The method according to claim 32, wherein said ErbB family receptor is the epidermal growth factor receptor (EGFR).

34. The method according to claim 32, wherein inhibiting RHBDL4 comprises introducing siRNA against RHBDL4 into said system.

35. The method according to claim 34, wherein said siRNA comprises the sequence of at least one of SEQ ID NO:1, SEQ ID NO:2 or SEQ ID NO:3.

36. The method according to claim 1, further comprising the step of assaying the effect of said modulator on RHBDL4 protease activity.

37. The method according to claim 36, wherein the effect on said RHBDL4 protease activity is determined according to claim 29.
